(19) United States
(12) Patent Application Publication
(10) Pub. No.: US 2009/0023763 A1
(43) Pub. Date: Jan. 22, 2009

(54) Condensed Pyridine Derivatives Useful as A2B Adenosine Receptor Antagonists

(76) Inventors: Bernat Vidal Juan, St. Cebria de Vallalta (ES); Paul Robert Eastwood, Rubi (ES); Jacob Gonzalez Rodriguez, Sant Feliu de Llobregat (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/578,386

(22) PCT Filed: Apr. 12, 2005
(86) PCT No.: PCT/EP2005/003818

§ 371 Date: Jun. 2, 2008

(30) Foreign Application Priority Data
Apr. 15, 2004 (ES) 200400919

(51) Int. Cl. A61K 31/506; A61P 11/06; A61P 9/10; A61P 37/08; A61P 29/00; A61P 1/00; A61P 35/00; A61K 31/444; A61K 31/437; A61P 3/10; C07D 401/06; C07D 471/04; C07D 409/14; C07D 405/14

(52) U.S. Cl. 514/274; 546/258; 546/118; 546/113; 544/333; 546/256; 544/316; 544/331; 514/275; 514/303; 514/300; 514/334; 514/333

(57) ABSTRACT

New antagonists of the A_{2B} adenosine receptor represented by formula (I) are disclosed; as well as processes for their preparation, pharmaceutical compositions comprising them and their use in therapy as antagonists of the A_{2B} adenosine receptor.

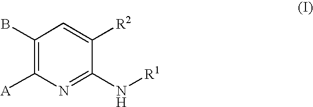

The present invention relates to new antagonists of the A_{2B} adenosine receptor. These compounds are useful in the treatment, prevention or suppression of diseases and disorders known to be susceptible to improvement by antagonism of the A_{2B} adenosine receptor, such as asthma, allergic diseases, inflammation, atherosclerosis, hypertension, gastrointestinal tract disorders, cell proliferation disorders, diabetes mellitus and autoimmune diseases.

Adenosine regulates several physiological functions through specific cell membrane receptors, which are members of the G-protein coupled receptor family. Four distinct adenosine receptors have been identified and classified: A₁, A_{2A}, A_{2B} and A₃.

The A_{2B} adenosine receptor subtype (see Feoktistov, I., Biaggioni, I. Pharmacol. Rev. 1997, 49, 381-402) has been identified in a variety of human and murine tissues and is involved in the regulation of vascular tone, smooth muscle growth, angiogenesis, hepatic glucose production, bowel movement, intestinal secretion, and mast cell degranulation.

In view of the physiological effects mediated by adenosine receptor activation, several A_{2B} receptor antagonists have been recently disclosed for the treatment or prevention of, asthma, bronchoconstriction, allergic diseases, hypertension, atherosclerosis, reperfusion injury, myocardial ischemia, retinopathy, inflammation, gastrointestinal tract disorders, cell proliferation diseases and/or diabetes mellitus. See for example WO03/063800, WO03/042214, WO 03/035639, WO02/42298, EP 1283056, WO 01/16134, WO 01/02400, WO01/60350 or WO 00/73307.

It has now been found that certain pyridine derivatives are novel potent antagonists of the A_{2B} adenosine receptor and can therefore be used in the treatment or prevention of these diseases.

Further objectives of the present invention are to provide a method for preparing said compounds; pharmaceutical compositions comprising an effective amount of said compounds; the use of the compounds in the manufacture of a medicament for the treatment of pathological conditions or diseases susceptible to improvement by antagonism of the A_{2B} adenosine receptor; and methods of treatment of pathological conditions or diseases susceptible to amelioration by antagonism of the A_{2B} adenosine receptor comprising the administration of the compounds of the invention to a subject in need of treatment.

Thus, the present invention is directed to the use of new pyridine derivatives of formula (I)

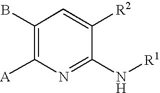

wherein:
A represents an optionally substituted monocyclic or polycyclic aryl or heteroaryl group,
B represents an optionally substituted monocyclic nitrogen-containing heterocyclic group, and either
a) R¹ represents a hydrogen atom and R² represents a group selected from —NH₂ and optionally substituted alkynyl groups
   or
b) R², R¹ and the —NH— group to which R¹ is attached form a moiety selected from the moieties of formulae (IIa), (IIb), (IIc), (IId) and (IIe):

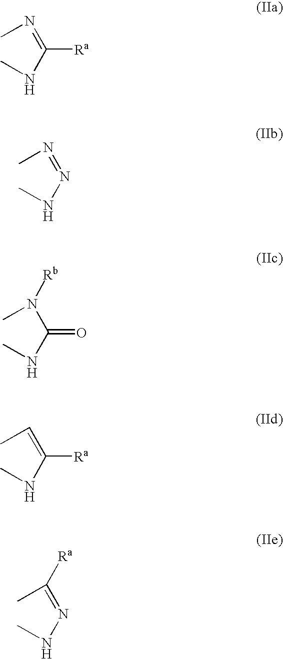

wherein:
R^{a} is selected from hydrogen atoms, halogen atoms and groups selected from optionally substituted alkyl, optionally substituted cycloalkyl, optionally substituted aryl, optionally substituted heteroaryl, —OR³, —SR³, —COOR³, —CONR³R⁴, —NR³R⁴, —NR³COR⁴ and —CN groups wherein R³ and R⁴ are independently selected from hydrogen atoms and lower alkyl or cycloalkyl groups.
R^{b} is selected from hydrogen atoms and groups selected from optionally substituted alkyl, optionally substituted cycloalkyl, optionally substituted aryl and optionally substituted heteroaryl groups,
in the manufacture of a medicament for the treatment of a pathological condition or disease susceptible to improvement by antagonism of the A_{2B} adenosine receptor.

In addition the compounds of formula (I)

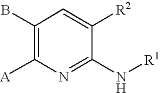

wherein A represents an optionally substituted monocyclic or polycyclic aryl or heteroaryl group, B represents an optionally substituted monocyclic nitrogen-containing heterocyclic group and either (a) R¹ represents a hydrogen atom and R² represents a group selected from —NH₂ and optionally substituted alkynyl groups or (b) R², R¹ and the —NH— group to which R¹ is attached form a moiety selected from the moieties of formulae (IIa), (IIb), (IIc) and (IId) wherein R^{a} and R^{b} are as hereinabove defined, are new and the invention is also directed to these compounds.

As used herein the terms alkyl or lower alkyl embraces optionally substituted, linear or branched hydrocarbon radicals having 1 to 8, preferably 1 to 6 and more preferably 1 to 4 carbon atoms. Preferred substituents on the alkyl groups are halogen atoms and hydroxy groups.

Examples include methyl, ethyl, n-propyl, i-propyl, n-butyl, sec-butyl and tert-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

As used herein the term alkynyl embraces optionally substituted, linear or branched radicals having 2 to 8, preferably 2 to 6 and more preferably 2 to 4 carbon atoms which contain 1 or 2, preferably 1 triple bond. The alkynyl groups are preferably unsubstituted or substituted by halogen atoms.

Examples include ethynyl, propyn-1-yl, propyn-2-yl, butyn-1-yl, butyn-2-yl, butyn-3-yl and 1-methyl-propyn-2-yl.

As used herein, the term cycloalkyl embraces saturated carbocyclic radicals and, unless otherwise specified, a cycloalkyl radical typically has from 3 to 7 carbon atoms.

Examples include cyclopropyl, cyclobutyl, cyclopentyl, cyclohexyl and cycloheptyl. When a cycloalkyl radical carries 2 or more substituents, the substituents may be the same or different. Preferred substituents on the cycloalkyl groups are halogen atoms and hydroxy groups.

As used herein, unless otherwise provided, the term aryl radical embraces typically a C₅-C₁₄ monocyclic or polycyclic aryl radical such as phenyl or naphthyl, anthranyl or phenanthryl. Optionally substituted phenyl is preferred. When an aryl radical carries 2 or more substituents, the substituents may be the same or different. Preferred substituents on the aryl radicals are halogen atoms and groups selected from —OR³, —SR³, —R³, and —NHR³. Halogen atoms are particularly preferred.

As used herein, unless otherwise provided, the term heteroaryl radical embraces typically a 5- to 14-membered ring system comprising at least one heteroaromatic ring and containing at least one heteroatom selected from O, S and N. A heteroaryl radical may be a single ring or two or more fused rings wherein at least one ring contains a heteroatom.

Examples of monocyclic heteroaryl radicals include pyridyl, pyrazinyl, pyrimidinyl, pyridazinyl, furyl, oxadiazolyl, oxazolyl, imidazolyl, thiazolyl, thiadiazolyl, thienyl, pyrrolyl, pyridinyl, triazolyl, imidazolidinyl and pyrazolyl radicals. Pyridyl, thienyl, furyl, pyridazinyl and pyrimidinyl radicals are preferred.

When a heteroaryl radical carries 2 or more substituents, the substituents may be the same or different. Preferred substituents on the heteroaryl radicals are halogen atoms and groups selected from —OR³, —SR³, —R³, and —NHR³.

As used herein, the term heterocyclic group embraces typically an heteroaromatic or non-aromatic, saturated or unsaturated C₃-C₁₀ carbocyclic ring, such as a 5, 6 or 7 membered radical, in which one or more, for example 1, 2, 3 or 4 of the carbon atoms, preferably 1 or 2, of the carbon atoms are replaced by a heteroatom selected from N, O and S. Non-saturated heterocyclyl radicals are preferred. A heterocyclic radical may be a single ring or two or more fused rings wherein at least one ring contains a heteroatom. When a heterocyclyl radical carries 2 or more substituents, the substituents may be the same or different.

Examples of monocyclic, nitrogen-containing heterocyclic radicals include pyridyl, pyrazinyl, pyrimidinyl, pyridazinyl, oxadiazolyl, oxazolyl, imidazolyl, thiazolyl, thiadiazolyl, pyrrolyl, pyridinyl, triazolyl, imidazolidinyl, pyrazolyl, piperidyl, pyrrolidyl, pyrrolinyl, piperazinyl, morpholinyl, thiomorpholinyl, pyrrolyl, pyrazolinyl, pirazolidinyl, quinuclidinyl, pyrazolyl, tetrazolyl, imidazolidinyl, imidazolyl, and 3-aza-tetrahydrofuranyl. Pyridyl, pyrimidinyl, pirazinyl and pyridazinyl are preferred radicals.

Where a heterocyclyl radical carries 2 or more substituents, the substituents may be the same or different. Preferred substituents on the aryl radicals are halogen atoms and group selected from —OR³, —SR³, —R³, and —NHR³. Halogen atoms are particularly preferred.

As used herein, some of the atoms, radicals, moieties, chains or cycles present in the general structures of the invention are “optionally substituted”. This means that these atoms, radicals, moieties, chains or cycles can be either unsubstituted or substituted in any position by one or more, for example 1, 2, 3 or 4, substituents, whereby the hydrogen atoms bound to the unsubstituted atoms, radicals, moieties, chains or cycles are replaced by chemically acceptable atoms, radicals, moieties, chains or cycles. When two or more substituents are present, each substituent may be the same or different.

As used herein, the term halogen atom embraces chlorine, fluorine, bromine or iodine atoms typically a fluorine, chlorine or bromine atom, most preferably chlorine or fluorine. The term halo when used as a prefix has the same meaning.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X-) is associated with the positive charge of the N atom. X- may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X- is preferably an anion selected from chloride, bromide, iodide, sulphate, nitrate, acetate, maleate, oxalate, succinate or trifluoroacetate. More preferably X- is chloride, bromide, trifluoroacetate or methanesulphonate.

As used herein, an N-oxide is formed from the tertiary basic amines or imines present in the molecule, using a convenient oxidising agent.

Preferred compounds of the invention are those wherein B represents an optionally substituted monocyclic, six-membered heterocyclic ring having one or two nitrogen atoms. More preferably B represents a group selected from optionally substituted pyridines, optionally substituted pyrimidines, optionally substituted pyridazines and optionally substituted pyridinones. Still more preferably B is unsubstituted or substituted with one group selected from —OR³, —SR³, —R³, and —NHR³.

In another embodiment of the present invention the group A represents an optionally substituted phenyl, furyl or thienyl group. Preferably the group A is unsubstituted or substituted with one group selected from halogen atoms and lower alkyl groups.

In a still more preferred embodiment of the present invention the group B represents a pyrimidinyl group and the group A represents a furyl group.

In an alternative embodiment of the present invention either R¹ represents a hydrogen atom or R², R¹ and the —NH— group to which R¹ is attached form a moiety selected from the moieties of formulae (IIc) and (IIe)

In still another embodiment of the present invention R² represents an —NH2 group or an optionally substituted alkynyl group.

In still another embodiment of the present invention R^{a} is selected from lower alkyl groups and cycloalkyl groups.

In still another embodiment of the present invention R^{b} is selected from the group consisting of lower alkyl groups and hydrogen atoms.

Particular individual compounds of the invention for their use in the manufacture of a medicament for the treatment of a pathological condition or disease susceptible to improvement by antagonism of the A_{2B} adenosine receptor include:
2-(3-Fluorophenyl)-3,4′-bipyridine-5,6-diamine
5-(3-Fluorophenyl)-6-pyridin-4-yl-3H-imidazo[4,5-b]pyridine
5-(3-Fluorophenyl)-2-methyl-6-pyridin-4-yl-3H-imidazo[4,5-b]pyridine
2-Cyclopropyl-5-(3-fluorophenyl)-6-pyridin-4-yl-3H-imidazo[4,5-b]pyridine
2-Ethyl-5-(3-fluorophenyl)-6-pyridin-4-yl-3H-imidazo[4,5-b]pyridine
5-(3-Fluorophenyl)-6-pyridin-4-yl-3H-[1,2,3]triazolo[4,5-b]pyridine
5-(3-Fluorophenyl)-6-pyridin-4-yl-1,3-dihydro-2H-imidazo[4,5-b]pyridin-2-one
5-Ethynyl-2-(3-fluorophenyl)-3,4′-bipyridin-6-amine
6-(3-Fluorophenyl)-5-pyridin-4-yl-1H-pyrrolo[2,3-b]pyridine
6-(2-Furyl)-5-pyrimidin-4-yl-1H-pyrazolo[3,4-b]pyridin-3-amine
N-[6-(2-furyl)-5-pyrimidin-4-yl-1H-pyrazolo[3,4-b]pyridin-3-yl]acetamide
5-(2-Furyl)-6-pyrimidin-4-yl-1,3-dihydro-2H-imidazo[4,5-b]pyridin-2-one
2-(2-thienyl)-3,4′-bipyridine-5,6-diamine
2-(2-furyl)-3,4′-bipyridine-5,6-diamine
6-(2-furyl)-5-[2-(methylthio)pyrimidin-4-yl]pyridine-2,3-diamine
6-(2-furyl)-5-pyrimidin-4-ylpyridine-2,3-diamine
6-Pyridin-4-yl-5-(2-thienyl)-1,3-dihydro-2H-imidazo[4,5-b]pyridin-2-one
2-ethoxy-5-(2-furyl)-6-pyrimidin-4-yl-3H-imidazo[4,5-b]pyridine
5-(2-furyl)-6-pyrimidin-4-yl-3H-imidazo[4,5-b]pyridine
5-(2-furyl)-2-methyl-6-pyrimidin-4-yl-3H-imidazo[4,5-b]pyridine
5-(2-Furyl)-2-methyl-6-pyridin-4-yl-3H-imidazo[4,5-b]pyridine
2-cyclopropyl-5-(2-furyl)-6-pyrimidin-4-yl-3H-imidazo[4,5-b]pyridine
2-Cyclopropyl-5-(2-furyl)-6-pyridin-4-yl-3H-imidazo[4,5-b]pyridine
5-(2-Furyl)-6-pyridin-4-yl-3H-imidazo[4,5-b]pyridine
5-(2-furyl)-6-[2-(methylthio)pyrimidin-4-yl]-3H-imidazo[4,5-b]pyridine
5-(2-furyl)-1-methyl-6-pyrimidin-4-yl-1,3-dihydro-2H-imidazo[4,5-b]pyridin-2-one
6-(2-furyl)-5-pyrimidin-4-yl-1H-pyrazolo[3,4-b]pyridine
3-chloro-6-(2-furyl)-5-pyrimidin-4-yl-1H-pyrazolo[3,4-b]pyridine
3-ethoxy-6-(2-furyl)-5-pyrimidin-4-yl-1H-pyrazolo[3,4-b]pyridine
6-(2-furyl)-5-[2-(methylthio)pyrimidin-4-yl]-1H-pyrazolo[3,4-b]pyridin-3-amine
6-(2-furyl)-5-pyrimidin-4-yl-1,2-dihydro-3H-pyrazolo[3,4-b]pyridin-3-one
6-(2-furyl)-5-[2-(methylthio)pyrimidin-4-yl]-1H-pyrazolo[3,4-b]pyridine
6-(2-furyl)-5-(2-methoxypyrimidin-4-yl)-1H-pyrazolo[3,4-b]pyridine
N-cyclopropyl-4-[6-(2-furyl)-1H-pyrazolo[3,4-b]pyridin-5-yl]pyrimidin-2-amine
4-[6-(2-furyl)-1H-pyrazolo[3,4-b]pyridin-5-yl]-N-isopropylpyrimidin-2-amine
5-(2-ethoxypyrimidin-4-yl)-6-(2-furyl)-1H-pyrazolo[3,4-b]pyridine
6-(2-furyl)-5-(2-isopropoxypyrimidin-4-yl)-1H-pyrazolo[3,4-b]pyridine
5-[2-(cyclohexyloxy)pyrimidin-4-yl]-6-(2-furyl)-1H-pyrazolo[3,4-b]pyridine
6-(2-furyl)-N-isobutyl-5-pyrimidin-4-yl-1H-pyrazolo[3,4-b]pyridin-3-amine
N-{6-(2-furyl)-5-[2-(methylthio)pyrimidin-4-yl]-1H-pyrazolo[3,4-b]pyridin-3-yl}acetamide
6-(3-fluorophenyl)-5-pyrimidin-4-yl-1H-pyrazolo[3,4-b]pyridin-3-amine
6-(3-fluorophenyl)-5-pyrimidin-4-yl-1H-pyrazolo[3,4-b]pyridine
6-(2-furyl)-5-pyrimidin-4-yl-1H-pyrrolo[2,3-b]pyridine
2-(3-fluorophenyl)-6-(2-furyl)-5-pyrimidin-4-yl-1H-pyrrolo[2,3-b]pyridine
6-(2-furyl)-2-phenyl-5-pyrimidin-4-yl-1H-pyrrolo[2,3-b]pyridine
6-(5-bromo-2-furyl)-3-chloro-5-pyrimidin-4-yl-1H-pyrazolo[3,4-b]pyridine
5-(5-Bromo-2-furyl)-6-pyrimidin-4-yl-1,3-dihydro-2H-imidazo[4,5-b]pyridin-2-one
6-(2-furyl)-5-pyrimidin-4-yl-1H-pyrazolo[3,4-b]pyridin-3-amine
N-[6-(2-furyl)-5-pyrimidin-4-yl-1H-pyrazolo[3,4-b]pyridin-3-yl]acetamide

Compounds of general formula (I) and in particular those wherein A, B, R^{a}, R^{b} and R³ are as hereinabove defined and either:
R¹ represents a hydrogen atom and R² represents a —NH₂ group, or
R², R¹ and the —NH— group to which R¹ is attached represent a moiety selected from (IIa), (IIb) and (IIc)
   may be prepared following the synthetic scheme depicted in FIG. 1.

Compounds of general formula (If) are prepared in several steps starting with the halogenation of 6-halopyridine derivatives (III) using reagents such as bromine or N-halosuccinimide in polar aprotic solvents such as DMF and at temperatures ranging from 0° C. to 100° C., to yield 5,6-dihalo-2-aminopyridines (not shown). These products are in turn nitrated in a two step process involving nitration of the amino group in a mixture of sulphuric and nitric acid in a temperature range between −10° C. and 0° C. followed by a sulphuric acid promoted rearrangement of the nitro group to produce compounds of formula (IV).

Regioselective Suzuki-type coupling of (IV) with a boronic acid or boronate derivative using a palladium catalyst such as tetrakis(triphenylphosphine)palladium(0) or [1,1′-bis(diphenylphosphino)ferrocene]palladium(II) dichloride dichloromethane complex (1:1) in solvents such as toluene or dioxane in the presence of an aqueous solution of a base such as sodium or caesium carbonate and at a temperature between 25° C. and 110° C. provides compounds of general formula (V).

Compounds of general formula (XXIII) are prepared from compounds of general formula (XXII) using the general Suzuki coupling procedure described above. Bromination using similar conditions as used in the preparation of (IV) provides compounds of general formula (V).

A further Suzuki-type coupling using (V) with a corresponding boronic acid or boronate derivative under the standard procedures for Pd catalyzed reactions described above provides the 2-amino-3-nitropyridines (VI). Reduction of the nitro group using standard hydrogenation conditions in the presence of hydrogen and using palladium on carbon as a catalyst provides the diamino derivatives (If).

Treatment of compounds of formula (If) with acylating agents such as anhydrides, acid chlorides or acylcarbonates in a polar organic solvent such as THF and in the presence of a convenient organic base (such as triethylamine) or inorganic base yields compounds of formula (XXI) which can be converted into the compounds of formula (Ia) by acid (for example acetic acid) or base (for example sodium hydroxide) catalyzed cyclization at temperatures ranging from 70° C. to 200° C.

Alternatively, diamino derivatives (If) can be cyclized to the imidazopyridines (Ia) by heating in neat trialkylorthoester or in an acetic acid solution of the orthoester derivatives and at a temperature between 70° C. and 200° C.

Following other synthetic pathways, treatment of (If) with carbonylating agents such as carbonyldiimidazole in polar aprotic solvents such as dimethylformamide and heating at temperatures between 50° C. and 200° C. provides the imidazolone compounds (1c).

Treatment of (If) either with organic nitrites such as 3-methylbutyl nitrite in organic solvents such as dioxane at temperatures ranging from 25° C. to 110° C. or with inorganic nitrites such as sodium nitrite in mixtures of water and acetic acid from 0° C. to 100° C. provides the triazolo derivatives (Ib).

Compounds of general formula (I) and in particular those wherein A, B and R^{a} are as hereinabove defined and either:
R¹ represents a hydrogen atom and R² represents an optionally substituted alkynyl group, or
R², R¹ and the —NH— group to which R¹ is attached represent a compound of formula (IId)
   may be prepared following the synthetic scheme depicted in FIG. 2.

The compounds are prepared from 5,6-dihaloaminopyridines (VII) by sequential Suzuki-type couplings using the corresponding boronic acids or boronates of A and B and using a palladium catalyst such as tetrakis(triphenylphosphine)palladium (0) or [1,1′-bis(diphenylphosphino)ferrocene]palladium(II) dichloride dichloromethane complex (1:1) in organic solvents such as toluene or dioxane in the presence of an aqueous solution of a base such as sodium or caesium carbonate and at a temperature ranging from 25° C. to 110° C. to give the aminopyridines of formula (IX). Further halogenation using reagents such as Br₂ or N-halosuccinimide in polar aprotic solvents such as DMF and at temperatures ranging from 0° C. to 100° C., followed by a Sonogashira-type coupling provides the alkynyl derivatives (Ih). Typically Sonogashira coupling takes place in the presence of the alkynyl derivative of R^{a} in a solvent that is inert to the reaction conditions such as THF, using an organic base, preferably triethylamine, and catalytic quantities of a copper salt (preferably copper (I) iodide) and a palladium derivative (such as dichlorobis(triphenylphosphine)palladium (II)). The temperature of the reaction is in the range of 70° C. to 150° C. These compounds can be converted into compounds of formula (Id) by cyclization mediated by the use of a suitable catalyst e.g. a copper salt (preferably copper (I) iodide) or a palladium derivative in polar aprotic solvents such as dimethylformamide and at a temperature ranging from 70-150° C.

Another alternative method to promote the cyclisation of (Ih) to (Id) consists in the use of a suitable base, for example potassium tert-butoxide, in a polar aprotic solvent such as dimethylformamide or 1-methyl-2-pyrrolidinone at temperatures ranging from 60-100° C.

Compounds of general formula (I) and in particular those wherein A, B and R^{a} are as hereinabove defined and R², R¹ and the —NH— group to which R¹ is attached represent a moiety selected from (IIc) and (IIe), may be prepared following the synthetic scheme depicted in FIG. 3.

The aldehydes of formula (XI) are reacted with the halomethyl derivatives of formula (XII) to yield ketones of formula (XIII) either via cyanohydrin intermediates or in a two step process involving the addition of an organometallic derivative of (XII), preferably a magnesium or zinc derivative, followed by oxidation of the resulting alcohol using oxidizing agents such as manganese (IV) oxide.

Alternatively ketones of formula (XIII) may be obtained by condensation of ethyl esters of formula (XIV) with compounds of formula (XX). This reaction is conveniently carried out in the presence of an organic base such as lithium bis(trimethylsilyl)amide at temperatures ranging from −10° C. to about 50° C. in an organic aprotic solvent, preferably tetrahydrofuran or diethyl ether.

Ketones of formula (XIII) may be reacted with neat N,N-dimethylformamide dialkyl acetal, such as dimethylacetal, at a temperature ranging from room temperature to 150° C. to yield dimethylamino α,β unsaturated ketones of formula (XV) which can be converted into the 2-oxo-1,2-dihydropyridine-3-carbonitriles of formula (XVI) by cyclization in the presence of cyanoacetamide using alkoxides such as sodium methoxide in polar aprotic solvents such as dimethylformamide and at temperatures ranging from 50° C. to 150° C. These compounds may be converted into the 2-chloronicotinonitriles of formula (XVII) by treatment of the resulting pyridone (XVI) with chlorinating agents such as POCl₃, PCl₅ or PhPOCl₂ or by using a combination of such reagents.

In one synthetic pathway 2-chloronicotinonitriles of formula (XVII) are reacted with hydrazine in a convenient organic solvent that does not interfere with the reaction such as ethanol at a temperature ranging from 25° C. to 150° C. to provide compounds of general formula (Ie). Further acylation using acid chlorides or anhydrides in the presence of a base such as triethylamine in solvents such as dichloromethane, or using neat pyridine as solvent, at temperatures ranging from 25° C. to 170° C. provides amides (Ie2).

Treatment of (Ie) with an aliphatic or aromatic aldehyde in a suitable solvent such as dichloroethane or methanol with an acid catalyst such as acetic acid in the presence of, or followed by treatment with, a suitable reducing agent such as sodium borohydride or sodium triacetoxy borohydride leads to products of type (Ie3).

Deamination of (Ie) by diazotization using sodium nitrite in an acidic medium such as a mixture of glacial acetic acid and hydrochloric acid at a temperature in the range of 0-5° C., followed by treatment with a suitable reducing agent such as hypophosphorous acid, provides (Ie4).

In another synthetic pathway 2-chloronicotinonitriles of formula (XVII) may be reacted with a saturated solution of ammonia in an organic solvent, preferably ethanol, at a temperature ranging from 25° C. to 150° C. to yield compounds of formula (XVIII). Hydrolisis of compounds (XVIII) to the carboxylic acid of formula (XIX) can be achieved with a base such as potassium hydroxide in aqueous or organic solvents such as ethylene glycol and at a temperature between 50° C. and 200° C. Alternatively this conversion can be achieved by heating (XVIII) in an aqueous acidic medium such as 6M aqueous sulphuric acid. Compounds (XIX) may be subjected to Curtius rearrangement by formation of an acyl azide using reagents such as diphenylphosphoryl azide (or sodium azide with activated acid) in an organic solvent compatible with these reaction conditions (e.g. dioxane) then heating the reaction mixture at a temperature between 50° C. and 200° C., with in situ formation of the target pyridoimidazolone ring yielding compounds of formula (Ic).

Carboxylic acid (XIX) can be converted to pyridine (IX) by decarboxylation in solvents such as quinoline in the presence of a suitable catalyst, such as copper, at temperatures ranging from 200-250° C., with or without the use of microwave irradiation.

Alternative general synthetic methods are depicted in FIG. 4.

Pyridone (XVI) can be converted to acid (XXIV) by hydrolysis of the nitrile functionality using a suitable inorganic base such as sodium or potassium hydroxide, with or without aqueous hydrogen peroxide, in a suitable solvent such as water, methanol or ethylene glycol at temperatures ranging from 40-160° C. Treatment of (XXIV) with a suitable chlorinating agent such as phosphorous oxychloride, with or without the use of a solvent such as dimethylformamide, at temperatures ranging from 90-120° C., followed by evaporation and treatment of the crude mixture with a suitable alcohol, such as methanol, leads directly to chloro esters of type (XXVIII). Treatment of (XXVIII) with a hydrazine, such as hydrazine monohydrate or (4-methoxybenzyl)hydrazine in a suitable solvent such as ethanol at temperatures ranging from 60-100° C. provides cyclised derivatives of type (XXIX). Reaction of (XXIX) with a suitable chlorinating agent, such as phosphorous oxychloride, at temperatures ranging from 90-120° C. gives rise to derivatives of type (Ii). Alternatively, in the case that (XXIX) has a suitable protecting group (for example PG=4-methoxybenzyl) then treatment of (XXIX) with a suitable base, such as sodium hydride, in a polar aprotic solvent, such as dimethylformamide, followed by the addition of an alkylating agent such as an alkyl bromide or iodide followed by removal of the protecting group using, for example, an acid such as trifluoroacetic acid in the presence of a cation scavenger, such as thioanisole, gives rise to molecules of type (Ij).

Hydrolysis of the ester moiety of (XXVIII) using a suitable base such as aqueous sodium or potassium hydroxide in a solvent such as ethanol or methanol at temperatures ranging from 0-30° C. leads to carboxylic acids of type (XXV). These compounds may be subjected to Curtius rearrangement by formation of an acyl azide using reagents such as diphenylphosphoryl azide (or sodium azide with activated acid) in tertiary butanol in the presence of an organic base such as triethylamine then heating the reaction mixture at a temperature between 50° C. and 200° C. to give the Boc-protected derivatives which upon treatment with an acid such as trifluoroacetic acid give rise to compounds of type (XXVI). Compounds of general formula (XXVI) can be transformed to compounds of general formula (If by reaction with ammonia using a copper salt, such as copper (I) chloride, as a catalyst at a temperature ranging from 50° C. to 200° C.

Cyanopyridine (XVII) reacts with conveniently protected amines, such as 4-methoxybenzylamine or 2,4-dimethoxybenzylamine, in the presence of a base such as triethylamine in a suitable solvent such as ethanol with or without the influence of microwave irradiation at temperatures ranging from 60-200° C. to give substituted derivatives of type (XXX). Hydrolisis of compounds (XXX) to the carboxylic acid of formula (XXXI) can be achieved with a base such as potassium hydroxide in aqueous or organic solvents such as ethylene glycol and at a temperature ranging from 50° C. to 200° C. These compounds may be subjected to Curtius rearrangement by formation of an acyl azide using reagents such as diphenylphosphoryl azide (or sodium azide with activated acid) in an organic solvent compatible with these reaction conditions (e.g. dioxane) then heating the reaction mixture at a temperature between 50° C. and 200° C., with in situ formation of the target pyridoimidazolone ring yielding compounds of formula (XXXII). Treatment of compounds of type (XXXII) with a suitable base, such as sodium hydride or potassium carbonate, in a polar aprotic solvent, such as dimethylformamide or dimethylsulfoxide, followed by the addition of an alkylating agent such as an alkyl bromide or iodide followed by removal of the amine protecting group by using, for example, an acid such as trifluoroacetic acid in the presence of a cation scavenger such as thioanisole at temperatures ranging from 0-100° C. gives rise to molecules of type (Ic2).

Adenosine 2B Receptor Subtype Functional Cellular cAMP Assay

The assay was carried out using CHO-K1 transfected with human recombinant A_{2B} receptor and a commercial EIA kit (Amersahm, RPN225). Cells were seeded in 96 well plates at 10.000 cells/well. After 24 h, plates were placed on ice for 5 minutes, the medium was removed, and all wells were rinsed twice with 100 μl of incubation medium (Hepes 25 mM, DMEM-F12). After washing, Rolipram (30 μM) and antagonists were added in 100 μl of incubation medium, and the plates were incubated for 15 minutes at 37° C. NECA was then added to reach a final concentration of 10 μM and the plates were incubated for another 15 minutes at 37° C. After incubation, medium was removed from all wells, 200 μl of lysis buffer (reactive 1B from Amersham RPN225) were added, and the plates were incubated 10 minutes at room temperature with slight agitation. After lysis, 100 μl of the lysate were transferred to a plate pretreated with anti-rabbit antibody, 100 μl of rabbit anti-cAMP serum were added to the wells and the plates were incubated for 2 h at 4° C. Peroxidase-coupled cAMP was then added, and the plates incubated for 1 hour at 4° C. Plates were then washed 4 times with 100 μl of buffer (washing buffer, Amersham RPN225). After washing, 150 μl of peroxidase substrate were added to the wells and the plates were incubated for 1 hour at room temperature. Finally, 100 μl of 1 M sulphuric acid were added to stop the reaction and the OD was measured at 450-495 nm.

Functional Kᵢ was calculated using the following formula (Cheng Y. C. And Prusoff W. H. Biochem. Pharmacol 1973, 22, 3099-3108): Kᵢ (cAMP, nM)=[IC₅₀/(1+([C]/K_{d}))], where IC₅₀ is the IC₅₀ for the test compound, [C] is the total NECA concentration and K_{d} is the EC₅₀ for NECA.

The compounds of formula (I) have been tested according to the assay described above and have shown to be potent inhibitors of the A_{2B} adenosine receptor subtype. Preferred pyridine derivatives of the invention possess a functional Kᵢ value for the inhibition of A_{2B} (determined as defined above) of less than 200 nM, preferably less than 50 nM, more preferably less than 10 nM and still more preferably less than 6 nM.

The pyridine derivatives of the invention are useful in the treatment or prevention of diseases known to be susceptible to improvement by treatment with an antagonist of the A_{2B} adenosine receptor. Such diseases are, for example, asthma, bronchoconstriction, allergic diseases, inflammation, reperfusion injury, myocardial ischemia, atherosclerosis, hypertension, retinopathy, diabetes mellitus, inflammation, gastrointestinal tract disorders, and/or autoimmune diseases. Examples of autoimmune diseases which can be treated or prevented using the compounds of the invention are Addison's disease, autoimmune hemolytic anemia, Crohn's disease, Goodpasture's syndrome, Graves disease, Hashimoto's thyroiditis, idiopathic thrombocytopenic purpura, insulin-dependent diabetes mellitus, multiple sclerosis, myasthenia gravis, pemphigus vulgaris, pernicious anemia, poststreptococcal glomerulonephritis, psoriasis, rheumatoid arthritis, scleroderma, Sjogren's syndrome, spontaneous infertility, and systemic lupus erythematosus.

Accordingly, the pyridine derivatives of the invention and pharmaceutically acceptable salts thereof, and pharmaceutical compositions comprising such compound and/or salts thereof, may be used in a method of treatment of disorders of the human or animal body which comprises administering to a subject requiring such treatment an effective amount of pyridine derivative of the invention or a pharmaceutically acceptable salt thereof.

The present invention also provides pharmaceutical compositions which comprise, as an active ingredient, at least a pyridine derivative of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application. Preferably the compositions are made up in a form suitable for oral, topical, nasal, rectal, percutaneous injectable administration or inhalation.

The pharmaceutically acceptable excipients which are admixed with the active compound or salts of such compound, to form the compositions of this invention are well-known per se and the actual excipients used depend inter alia on the intended method of administering the compositions.

Compositions of this invention are preferably adapted for injectable and oral administration. In this case, the compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Effective doses are normally in the range of 2-2000 mg of active ingredient per day. Daily dosage may be administered in one or more treatments, preferably from 1 to 4 treatments, per day.

The syntheses of the compounds of the invention and of the intermediates for use therein are illustrated by the following Examples (1 to 49) including Preparation Examples (Intermediates 1 to 22) which do not limit the scope of the invention in any way.

¹H Nuclear Magnetic Resonance Spectra were recorded on a Varian Gemini 300 spectrometer. Melting points were recorded using a Büchi B-540 apparatus. The chromatographic separations were obtained using a Waters 2795 system equipped with a Symmetry C18 (2.1×100 mm, 3.5 mm) column. As detectors a Micromass ZMD mass spectrometer using ES ionization and a Waters 996 Diode Array detector were used. The mobile phase was formic acid (0.46 ml), ammonia (0.115 ml) and water (1000 ml) (A) and formic acid (0.4 ml), ammonia (0.1 ml), methanol (500 ml) and acetonitrile (500 ml) (B): initially from 0% to 95% of B in 20 min, and then 4 min. with 95% of B. The reequilibration time between two injections was 5 min. The flow rate was 0.4 ml/min. The injection volume was 5 μl. Diode array chromatograms were processed at 210 nm.

### PREPARATION EXAMPLES

### Intermediate 1

### 5,6-Dibromopyridin-2-amine

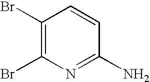

To a stirred solution of 2-amino-6-bromopyridine (4.0 g, 23.1 mmol) in N,N-dimethylformamide (55 mL) was added N-bromosuccinimide (4.12 g, 23.2 mmol) in portions over 30 minutes. After stirring overnight the mixture was poured into water and the precipitate was filtered, washed with water and dried to give the title compound (Intermediate 1) (4.98 g, 86%) as a white solid.

δ ¹H NMR (CDCl₃): 4.63 (s, 2H), 6.33 (d, 1H), 7.52 (d, 1H).

ESI/MS (m/e, %): 251 [(M+1)⁺, 100].

### Intermediate 2

### Step a:

### 5,6-Dibromo-N-nitropyridin-2-amine

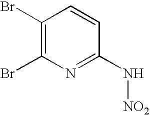

5,6-Dibromopyridin-2-amine (intermediate 1) (7.87 g, 31.2 mmol) was added in portions with stirring to cooled (0° C.) concentrated sulphuric acid (32 mL). Concentrated nitric acid (3.94 mL, 63 mmol) was added dropwise keeping the mixture at −10° C. The mixture was then warmed to 0° C. over 25 minutes, stirred at 0° C. for 30 minutes then poured onto ice. Maintaining the temperature at 0-5° C., the solution was treated with concentrated aqueous ammonia solution until a pH of 5 was reached. The precipitate was filtered, washed with water and dried to give the title compound (8.9 g, 96%) as a yellow solid.

δ ¹H NMR (DMSO): 7.80 (d, 1H), 8.30 (d, 1H).

ESI/MS (m/e, %): 296 [(M+1)⁺, 100].

### Step b:

### 5,6-Dibromo-3-nitropyridin-2-amine

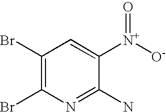

5,6-Dibromo-N-nitropyridin-2-amine was added in portions over 45 minutes to stirred concentrated sulphuric acid (30 mL). After the addition the mixture was stirred at room temperature for one hour then poured onto crushed ice. The mixture was taken to pH 9 with concentrated aqueous ammonia solution maintaining the internal temperature at 0° C. The solid was filtered, washed repeatedly with 1% aqueous ammonia solution and dried to give the title compound (7.33 g, 64%).

δ ¹H NMR (DMSO): 8.30 (s, 2H), 8.60 (s, 1H).

ESI/MS (m/e, %): 296 [(M+1)⁺, 100].

### Step c:

### 5-Bromo-6-(3-fluorophenyl)-3-nitropyridin-2-amine

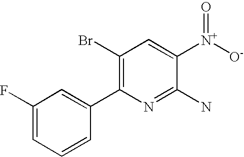

A mixture of 5,6-dibromo-3-nitropyridin-2-amine (2.93 g, 9.87 mmol), 3-fluorophenylboronic acid (1.38 g, 9.87 mmol), tetrakis(triphenylphosphine)palladium(0) (0.34 g) and 2M aqueous sodium carbonate solution (9.84 mL) in toluene (50 mL) and methanol (5 mL) was stirred under an atmosphere of argon and heated to 90° C. The mixture was stirred overnight then partitioned between ethyl acetate and water. The organic layer was dried (MgSO₄) and evaporated. Flash chromatography (15:1 hexanes/EtOAc) furnished the title compound (0.67 g, 22%) as a yellow solid.

δ ¹H NMR (DMSO): 7.25-7.6 (m, 4H), 8.10 (s, 2H), 8.62 (s, 1H).

ESI/MS (m/e, %): 312 [(M+1)⁺, 100].

### Step d:

### 2-(3-Fluorophenyl)-5-nitro-3,4′-bipyridin-6-amine (Intermediate 2)

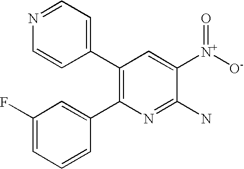

A mixture of 5-bromo-6-(3-fluorophenyl)-3-nitropyridin-2-amine (0.35 g, 1.12 mmol), 4-(4,4,5,5-tetramethyl-1,3,2-dioxaborolan-2-yl)pyridine (0.46 g, 2.24 mmol), [1,1′-bis(diphenylphosphino)ferrocene]palladium(II) dichloride dichloromethane complex (1:1) (55 mg) and 2M aqueous caesium carbonate solution (1.5 mL) in dioxane (14 mL) was heated to 90° C. under an atmosphere of Argon. The mixture was stirred overnight then partitioned between ethyl acetate and water. The organic layer was dried (MgSO₄) and evaporated. Flash chromatography (3:1 hexanes/EtOAc) furnished 2-(3-fluorophenyl)-5-nitro-3,4′-bipyridin-6-amine (intermediate 2) (0.34 g, 97%) as a yellow solid.

δ ¹H NMR (CDCl₃): 7.00-7.30 (m, 7H), 8.57 (d, 2H).

ESI/MS (m/e, %): 311 [(M+1)⁺, 100].

### Intermediate 3

### Step a:

### 5-Bromo-6-(3-fluorophenyl)pyridin-2-amine

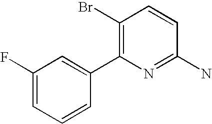

To a solution of 5,6-dibromopyridin-2-amine (Intermediate 1) (2.0 g, 7.94 mmol) and 3-fluorophenylboronic acid (1.11 g, 7.94 mmol) in toluene (40 mL) and methanol (4 mL) was added 2M aqueous sodium carbonate solution (7.94 mL). The mixture was purged with argon and then tetrakis(triphenylphosphine)palladium(0) (0.275 g, 0.24 mmol) was added. The mixture was heated to reflux and left to stir overnight. The mixture was then cooled, diluted with ethyl acetate and washed with water, brine, dried (MgSO₄) and evaporated to give the crude title compound (2.35 g) that was used directly.

δ ¹H NMR (CDCl₃): 4.60 (s, 2H), 6.40 (d, 1H), 7.0-7.50 (m, 4H), 7.62 (d, 1H).

ESI/MS (m/e, %): 267 [(M+1)⁺, 100].

### Step b:

### 2-(3-Fluorophenyl)-3,4′-bipyridin-6-amine

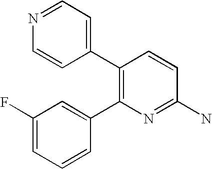

A mixture of 5-bromo-6-(3-fluorophenyl)pyridin-2-amine (1.50 g, 5.62 mmol), 4-(4,4,5,5-tetramethyl-1,3,2-dioxaborolan-2-yl)pyridine (2.30 g, 11.24 mmol), [1,1′-bis(diphenylphosphino)ferrocene]palladium(II) dichloride dichloromethane complex (1:1) (300 mg) and 2M aqueous caesium carbonate solution (8.4 mL) in dioxane (60 mL) was heated to 90° C. under an atmosphere of Argon. The mixture was stirred overnight then partitioned between ethyl acetate and water. The organic layer was dried (MgSO₄) and evaporated. Flash chromatography (100:1 dichloromethane/methanol) furnished the title compound (1.14 g, 77%) as a white solid.

δ ¹H NMR (CDCl₃): 4.65 (s, 1H), 6.95-7.25 (m, 5H), 7.45 (m, 2H), 8.40 (m, 2H).

ESI/MS (m/e, %): 266 [(M+1)⁺, 100].

### Step c:

### 5-Bromo-2-(3-fluorophenyl)-3,4′-bipyridin-6-amine

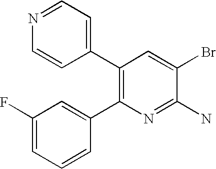

To a solution of 2-(3-fluorophenyl)-3,4′-bipyridin-6-amine (0.20 g, 0.75 mmol) in N,N-dimethylformamide (2 mL) was added N-bromosuccinimide (0.14 g, 0.79 mmol) and the mixture was stirred at room temperature overnight. The solution was poured into water and extracted with ethyl acetate. The organic layer was washed with brine, dried (MgSO₄) and evaporated. The residue was purified by flash chromatography (4:1 hexanes/ethyl acetate to 2:1 hexanes/ethyl acetate) to give the title compound (0.18 g, 70%) as an off-white solid.

δ ¹H NMR (CDCl₃): 5.17 (s, 2H), 6.90-7.30 (m, 6H), 7.78 (s, 1H), 8.45 (d, 2H).

ESI/MS (m/e, %): 344 [(M+1)⁺, 100].

### Step d:

### 2-(3-Fluorophenyl)-5-[(trimethylsilyl)ethynyl]-3,4′-bipyridin-6-amine (Intermediate 3)

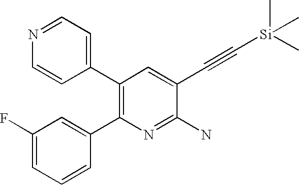

To a solution of 5-bromo-2-(3-fluorophenyl)-3,4′-bipyridin-6-amine (100 mg, 0.29 mmol) in tetrahydrofuran (0.3 mL) under an atmosphere of argon was added triethylamine (1.75 mL), copper(I) iodide (2.2 mg, 0.012 mmol), bis(triphenylphosphine)palladium(II) chloride (8.2 mg, 0.012 mmol) and trimethylsilylacetylene (57 mg, 0.58 mmol). The mixture was heated to 90° C. in a sealed tube and stirred overnight. The mixture was cooled, diluted with water and extracted with ethyl acetate. The organic layer was dried (MgSO₄) and evaporated to give 2-(3-fluorophenyl)-5-[(trimethylsilyl)ethynyl]-3,4′-bipyridin-6-amine (intermediate 3) as a brown solid.

δ ¹H NMR (CDCl₃): 0.3 (s, 9H), 5.20 (s, 2H), 6.9-7.4 (m, 6H), 7.60 (s, 1H), 8.43 (d, 2H).

ESI/MS (m/e, %): 362 [(M+1)⁺, 100].

### Intermediate 4

### Step a:

### 1-(2-Furyl)-2-pyrimidin-4-ylethanone

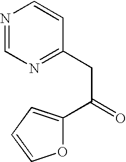

Lithium bis(trimethylsilyl)amide (1.0 M in hexanes, 50 mL) was added dropwise over 60 minutes to a solution of 4-methylpyrimidine (2.33 g, 24.8 mmol) and ethyl 2-furoate (3.85 g, 27.4 mmol) in tetrahydrofuran (20 mL) under an atmosphere of nitrogen. The mixture was stirred at ambient temperature for two hours then hexane (200 mL) was added and the precipitate was filtered. The solid was treated with saturated aqueous ammonium chloride solution, filtered and washed with water and dried in vacuo to give the title compound (8.62 g, 93%) as a yellow solid.

δ ¹H NMR (DMSO) showed a mixture of enol and keto tautomers: Keto tautomer: 4.39 (s, 2H), 6.75 (dd, 1H), 7.08 (m, 1H), 7.53 (dd, 1H), 7.61 (d, 1H), 8.04 (dd, 1H), 9.08 (d, 1H). Enol tautomer: 5.99 (s, 1H), 6.64 (dd, 1H), 7.04 (d, 1H), 7.85 (dd, 1H), 8.15 (d, 1H), 8.61 (s, 1H), 8.74 (d, 1H).

ESI/MS (m/e, %): 189 [(M+1)⁺, 100].

### Step b:

### (2Z)-3-(Dimethylamino)-1-(2-furyl)-2-pyrimidin-4-ylprop-2-en-1-one

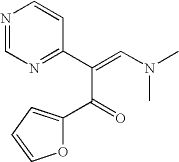

A suspension of 1-(2-furyl)-2-pyrimidin-4-ylethanone (8.62 g, 45.9 mmol) in N,N-dimethylformamide diethyl acetal (40 mL) was heated to reflux. The mixture was stirred for 2.5 hours then evaporated to give the title compound as a dark oil in quantitative yield.

δ ¹H NMR (CDCl₃): 3.0 (s, 6H), 6.40 (dd, 1H), 6.80 (m, 1H), 7.00 (m, 1H), 7.40 (m, 1H), 7.80 (m, 1H), 8.40 (d, 1H), 9.00 (s, 1H).

ESI/MS (m/e, %): 244 [(M+1)⁺, 100].

### Step c:

### 6-(2-Furyl)-2-oxo-5-pyrimidin-4-yl-1,2-dihydropyridine-3-carbonitrile

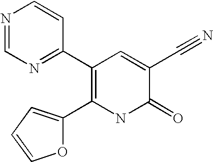

Sodium methoxide (5.88 g, 109 mmol) was added to a mixture (2Z)-3-(dimethylamino)-1-(2-furyl)-2-pyrimidin-4-ylprop-2-en-1-one (45.9 mmol) and 2-cyanoacetamide (4.65 g, 55.3 mmol) in dimethylformamide (110 mL) under an atmosphere of argon. The mixture was heated to 80° C. and stirred for two hours then concentrated under high vacuum at 65° C. Water was added to the residue and the pH adjusted to 4-5 with 5M aqueous hydrochloric acid. The precipitate was filtered and dried in vacuo to give the title compound (8.52 g, 70%) as an orange solid.

δ ¹H NMR (DMSO): 6.67 (dd, 1H), 7.13 (dd, 1H), 7.21 (dd, 1H), 7.71 (dd, 1H), 8.30 (s, 1H), 8.71 (d, 1H), 9.13 (d, 1H).

ESI/MS (m/e, %): 265 [(M+1)⁺, 100].

### Step d:

### 2-Chloro-6-(2-furyl)-5-pyrimidin-4-ylnicotinonitrile (Intermediate 4)

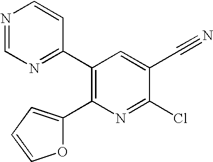

A suspension of 6-(2-furyl)-2-oxo-5-pyrimidin-4-yl-1,2-dihydropyridine-3-carbonitrile (3.74 g, 14.2 mmol) in phosphorus oxychloride (20 mL) was heated to reflux and stirred overnight. The mixture was evaporated and carefully neutralised with 4% aqueous sodium hydrogen carbonate solution. Ethyl acetate was added to the solution and, after stirring for five minutes, the mixture was filtered to remove an insoluble black solid. The organic layer was dried over MgSO₄, filtered and evaporated in vacuo to give 2-chloro-6-(2-furyl)-5-pyrimidin-4-ylnicotinonitrile (Intermediate 4) (2.5 g, 63%) as a brown solid.

δ ¹H NMR (DMSO): 6.65 (dd, 1H), 7.07 (dd, 1H), 7.66 (dd, 1H), 7.72 (dd, 1H), 8.60 (s, 1H), 8.94 (d, 1H), 9.27 (d, 1H).

ESI/MS (m/e, %): 283 [(M+1)⁺, 100].

### Intermediate 5

### Step a:

### 2-Amino-6-(2-furyl)-5-pyrimidin-4-ylnicotinonitrile

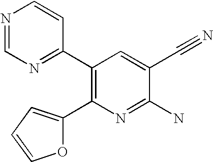

2-Chloro-6-(2-furyl)-5-pyrimidin-4-ylnicotinonitrile (Intermediate 4) (1.20 g, 4.25 mmol) and a saturated solution of ammonia in ethanol (60 mL) were heated in a sealed tube to 100° C. After four hours the mixture was cooled and evaporated. Flash chromatography (100:1 dichloromethane/methanol) gave the title compound (0.78 g, 70%) as a white solid.

δ ¹H NMR (CDCl₃): 5.40 (s, 2H), 6.43 (dd, 1H), 6.85 (dd, 1H), 7.10 (d, 1H), 7.35 (d, 1H), 8.00 (s, 1H), 8.62 (d, 1H), 9.27 (s, 1H).

ESI/MS (m/e, %): 264 [(M+1)⁺, 100].

### Step b:

### 2-Amino-6-(2-furyl)-5-pyrimidin-4-ylnicotinic acid (Intermediate 5)

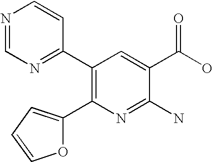

A suspension of 2-amino-6-(2-furyl)-5-pyrimidin-4-ylnicotinonitrile (0.52 g, 2.0 mmol) and potassium hydroxide (0.45 g, 8.1 mmol) in ethylene glycol (7 mL) was heated to 150° C. After stirring five hours the yellow solution was poured onto ice water and taken to pH 4.5 with 5M aqueous hydrochloric acid. The precipitate was filtered and dried in vacuo to give 2-amino-6-(2-furyl)-5-pyrimidin-4-ylnicotinic acid (Intermediate 5) (0.40 g, 72%) as a yellow solid.

δ ¹H NMR (DMSO): 6.59 (dd, 1H), 6.83 (dd, 1H), 7.19 (dd, 1H), 7.62 (m, 2H), 8.28 (s, 1H), 8.67 (d, 1H), 9.14 (d, 1H).

ESI/MS (m/e, %): 283 [(M+1)⁺, 100].

### Intermediate 6

### N-[6-amino-2-(3-fluorophenyl)-3,4′-bipyridin-5-yl]yclopropanecarboxamide

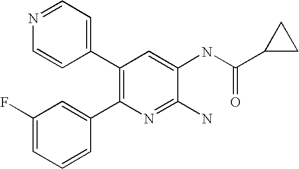

To a stirred solution of cyclopropanecarboxylic acid (30.7 mg, 0.35 mmol) in THF (5 mL) was added triethylamine (48 uL, 0.35 mmol) and the mixture was cooled to −10° C. Ethylchloroformate (38 mg, 0.35 mmol) was added dropwise and the mixture was stirred for 20 minutes and then a solution 2-(3-fluorophenyl)-3,4′-bipyridine-5,6-diamine (Example 1) (0.1 g, 0.35 mmol) in THF (4 mL) was added dropwise. The mixture was warmed to room temperature and stirred for two hours. The mixture was evaporated and partitioned between saturated aqueous sodium hydrogen carbonate solution and ethyl acetate. The organic layer was dried (MgSO₄) and evaporated to give a solid which was purified by flash chromatography (1:1 hexanes/ethyl acetate followed by 95:5 dichloromethane/methanol) give the title compound (Intermediate 6) (50 mg, 42%) as a white solid.

δ ¹H NMR (CDCl₃): 0.8-1.2 (m, 5H), 4.87 (s, 2H), 6.95-7.30 (m, 6H), 7.60 (s, 1H), 8.43 (d, 2H).

ESI/MS (m/e, %): 349 [(M+1)⁺, 100].

### Intermediate 7

### Step a:

### 5-Bromo-3-nitro-6-thien-2-ylpyridin-2-amine

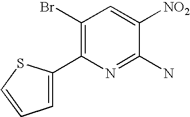

A mixture of 5,6-dibromo-3-nitropyridin-2-amine (0.300 g, 1.010 mmol), described in step b of intermediate 2,2-thienylboronic acid (0.123 g, 0.960 mmol), [1,1′-bis(diphenylphosphino)ferrocene]palladium(II) dichloride dichloromethane complex (1:1) (0.049 g, 0.006 mmol) and 2M aqueous caesium carbonate solution (1.5 mL) in dioxane (9 mL) was heated to 80° C. under an argon atmosphere for 18 hours. The mixture was then partitioned between ethyl acetate and water. The organic layer was dried (MgSO₄) and evaporated. The crude mixture was purified by flash chromatography (95:5 hexanes/ethyl acetate) to give the title compound (0.092 g, 30%).

δ ¹H NMR (CDCl₃): 7.12-7.20 (m, 1H), 7.57 (d, 1H), 8.34-8.36 (m, 1H), 8.65 (s, 1H).

ESI/MS (m/e, %): 301 [(M+1)⁺, 100].

### Step b:

### 5-Nitro-2-thien-2-yl-3,4′-bipyridin-6-amine (Intermediate 7)

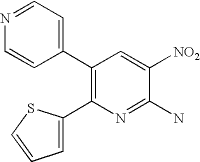

A mixture of 5-bromo-3-nitro-6-thien-2-ylpyridin-2-amine (0.093 g, 0.310 mmol) and 2-(4-pyridyl)-4,4,5,5-tetramethyl-1,3,2-borolane (0.083 g, 0.40 mmol), [1,1′-bis(diphenylphosphino)ferrocene]palladium(II) dichloride dichloromethane complex (1:1) (0.015 g, 0.019 mmol) and 2M aqueous caesium carbonate solution (0.5 mL) in dioxane (5 mL) was heated to 80° C. under an argon atmosphere for 18 hours. The mixture was then partitioned between ethyl acetate and water. The organic layer was dried (MgSO₄) and evaporated. The crude mixture was purified by flash chromatography (3:1 hexanes/ethyl acetate) to give the title compound (Intermediate 7) (0.092 g, 99%).

δ ¹H NMR (CDCl₃): 6.72-6.74 (m, 1H), 6.87 (dd, 1H), 7.29-7.42 (m, 2H), 7.44-7.47 (m, 1H), 8.31 (s, 1H), 8.67-8.70 (m, 2H).

ESI/MS (m/e, %): 299 [(M+1)⁺, 100].

### Intermediate 8

### Step a:

### 6-(2-Furyl)-3-nitropyridin-2-amine

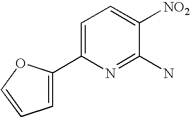

To a solution of 6-bromo-3-nitropyridin-2-amine (1.0 g, 4.6 mmol) and 2-furylboronic acid (0.76 g, 6.9 mmol) in dimethoxyethane (30 mL) and water (2 mL), potassium carbonate (0.58 g, 4.23 mmol) was added. The mixture was purged with Argon and then tetrakis(triphenylphosphine)palladium(0) (0.53 g; 0.46 mmol) was added. The mixture was heated at 80° C. for 16 hours. The mixture was then cooled, diluted with ethyl acetate and washed with water, brine, dried (MgSO₄) and evaporated. The crude mixture was purified by flash chromatography (3:1 hexanes/ethyl acetate) to give the title compound (0.43 g, 46%).

δ ¹H NMR (CDCl₃): 6.57-6.59 (m, 1H), 7.15-7.19 (m, 2H), 7.61 (s, 1H), 8.43 (d, 1H).

ESI/MS (m/e, %): 206 [(M+1)⁺, 100].

### Step b:

### 5-Bromo-6-(2-furyl)-3-nitropyridin-2-amine (Intermediate 8)

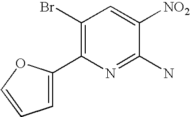

To a solution of 6-(2-furyl)-3-nitropyridin-2-amine (0.100 g, 0.49 mmol) in 2 mL of DMF at 0° C. under argon atmosphere, N-bromosuccinimide (0.083 g, 0.46 mmol) in portions was added. After 40 minutes at 0° C., the mixture was poured into ice-water and the precipitate formed was filtered off. The crude mixture was purified by HPLC (acetonitrile/water gradient) to give the title compound (Intermediate 8) (0.05 g, 22%).

δ ¹H NMR (CDCl₃): 6.60-6.62 (m, 1H), 7.66-7.72 (m, 2H), 8.67 (s, 1H).

ESI/MS (m/e, %): 285 [(M+1)⁺, 100].

### Intermediate 9

### 2-(2-Furyl)-5-nitro-3,4′-bipyridin-6-amine

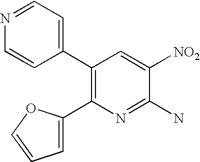

A mixture of 5-bromo-6-(2-furyl)-3-nitropyridin-2-amine (Intermediate 8) (0.220 g, 0.770 mmol), 2-(4-pyridyl)-4,4,5,5-tetramethyl-1,3,2-borolane (0.205 g, 1 mmol), [1,1′-bis(diphenylphosphino)ferrocene]palladium(II) dichloride dichloromethane complex (1:1) (0.028 g, 0.046 mmol) and caesium carbonate (0.0756 g, 2.31 mmol) in dioxane (8 mL) and water (2 mL) was heated to 80° C. under argon atmosphere for 18 hours. The mixture was then partitioned between ethyl acetate and water. The organic layer was dried (MgSO₄) and evaporated. The crude mixture was purified by flash chromatography (3:1 hexanes/ethyl acetate) to give the title compound (Intermediate 9) (0.250 g, 52%).

δ ¹H NMR (DMSO): 6.40-6.43 (m, 1H), 6.58 (d, 1H), 7.24-7.27 (m, 2H), 7.41-7.43 (m, 1H), 8.34 (s, 1H), 8.66-8.69 (m, 2H).

ESI/MS (m/e, %): 283 [(M+1)⁺, 100].

### Intermediate 10

### 2-Chloro-6-(2-furyl)-5-pyrimidin-4-ylnicotinic acid

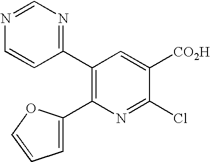

To a solution of methyl 2-chloro-6-(2-furyl)-5-pyrimidin-4-ylnicotinate (Intermediate 14) (9.21 g, 29.17 mmol) in 60 mL of ethanol was added 2M aqueous sodium hydroxide (30 mL). After 2 hours at room temperature, the solvent was evaporated and the crude mixture was diluted with water. The pH was taken to 6-7 and the precipitate formed was filtered off to give the title compound (Intermediate 10) (8.64 g, 98%).

δ ¹H NMR (DMSO): 6.60-6.63 (m, 1H), 6.97 (d, 1H), 7.59-7.67 (m, 2H), 8.31 (s, 1H), 8.88 (d, 1H), 9.26-9.28 (m, 1H).

ESI/MS (m/e, %): 302 [(M+1)⁺, 100].

### Intermediate 11

### 2-Chloro-6-(2-furyl)-5-pyrimidin-4-ylpyridin-3-amine

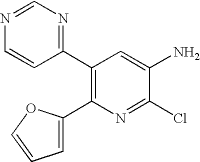

To a solution of 2-chloro-6-(2-furyl)-5-pyrimidin-4-ylnicotinic acid (8.64 g, 28.6 mmol) and triethylamine (4.4 mL, 31.46 mmol) in 80 mL of tert-butyl alcohol was added diphenylphosphoryl azide (8.66 g, 31.46 mmol). The mixture was heated to reflux for 3 hours and after cooling to room temperature, ethyl acetate was added. The organic layer was washed with 2M aqueous sodium hydroxide, water and brine, dried and evaporated to give tert-butyl 2-chloro-6-(2-furyl)-5-pyrimidin-4-ylpyridin-3-ylcarbamate (9.1 g, 86%). To a solution of tert-butyl 2-chloro-6-(2-furyl)-5-pyrimidin-4-ylpyridin-3-ylcarbamate (9.1 g, 24.43 mmol) in 90 mL of dichloromethane was added 28.5 mL (366.4 mmol) of trifluoroacetic acid. The mixture was stirred at room temperature for 2 hours and the solvent was evaporated. The crude mixture was partitioned between ethyl acetate and 4% aqueous sodium hydrogen carbonate and the organic layer was dried and evaporated. The crude mixture was purified by flash chromatography (1:3 hexanes/ethyl acetate) to give the title compound (Intermediate 11) (4.64 g, 70%).

δ ¹H NMR (DMSO): 6.03 (s, 2H), 6.45-6.47 (m, 2H), 7.26 (s, 1H), 7.31 (dd, 1H), 7.45-7.47 (m, 1H), 8.78 (d, 1H), 9.23 (d, 1H).

ESI/MS (m/e, %): [(M+1)⁺, 100].

### Intermediate 12

### Step a:

### 1-(2-furyl)-2-[2-(methylthio)pyrimidin-4-yl]ethanone

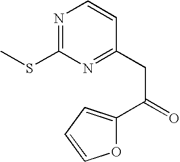

Lithium bis(trimethylsilyl)amide (1.0 M in hexanes, 100 mL, 100 mmol) was added dropwise over 60 minutes to a solution of 4-methyl-2-(methylthio)pyrimidine (7.02 g, 50.0 mmol) and ethyl 2-furoate (7.70 g, 55.0 mmol) in tetrahydrofuran (22 mL) under an atmosphere of nitrogen. The mixture was stirred at ambient temperature overnight then hexane (200 mL) was added and the precipitate was filtered. The solid was treated with saturated aqueous ammonium chloride solution, filtered and washed with water and dried. Purification by flash chromatography (8:2 ethyl acetate/hexanes to 5:1 ethyl acetate/hexanes) gave the title compound (10.32 g, 88%) as a yellow solid.

δ ¹H NMR (DMSO) showed a mixture of enol and keto tautomers: Keto tautomer: 2.42 (s, 3H), 4.35 (s, 2H), 6.75 (dd, 1H), 7.22 (d, 1H), 7.60 (dd, 1H), 8.05 (d, 1H), 8.60 (d, 1H). Enol tautomer: 2.42 (s, 3H), 6.18 (s, 1H), 6.70 (dd, 1H), 7.05 (m, 2H), 7.90 (d, 1H), 8.45 (d, 1H).

ESI/MS (m/e, %): 235 [(M+1)⁺, 100].

### Step b:

### (2Z)-3-(dimethylamino)-1-(2-furyl)-2-[2-(methylthio)pyrimidin-4-yl]prop-2-en-1-one

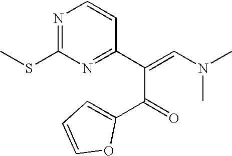

A suspension of 1-(2-furyl)-2-[2-(methylthio)pyrimidin-4-yl]ethanone (10.32 g, 44.0 mmol) in N,N-dimethylformamide diethyl acetal (50 mL) was heated to reflux. The mixture was stirred for 3 hours then evaporated in vacuo to give the title compound as a dark oil in quantitative yield.

δ ¹H NMR (CDCl₃): 8.13 (d, 1H), 7.76 (m, 1H), 7.42 (dd, 1H), 6.87 (dd, 1H), 6.68 (d, 1H), 6.44 (dd, 1H), 3.01 (s, 6H), 2.54 (s, 3H).

ESI/MS (m/e, %): 290 [(M+1)⁺, 100].

### Step c:

### 6-(2-furyl)-5-[2-(methylthio)pyrimidin-4-yl]-2-oxo-1,2-dihydropyridine-3-carbonitrile

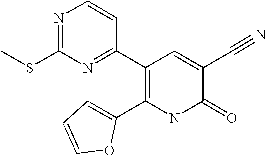

Sodium methoxide (5.38 g, 99.5 mmol) was added to a mixture of (2Z)-3-(dimethylamino)-1-(2-furyl)-2-[2-(methylthio)pyrimidin-4-yl]prop-2-en-1-one (41.5 mmol) and 2-cyanoacetamide (4.18 g, 49.8 mmol) in dimethylformamide (65 mL) under an atmosphere of argon. The mixture was heated to 80° C. and stirred for four hours then concentrated under high vacuum at 65° C. Water was added to the residue and the pH adjusted to 4-5 with 5M aqueous hydrochloric acid. The precipitate was filtered and dried in vacuo to give the title compound (10.14 g, 79%) as a yellow solid.

δ ¹H NMR (DMSO): 13.70 (s, 1H), 8.61 (d, 1H), 8.42 (s, 1H), 7.76 (dd, 1H), 7.24 (dd, 1H), 7.02 (d, 1H), 6.71 (dd, 1H), 2.38 (s, 3H).

ESI/MS (m/e, %): 311-[(M+1)⁺, 100].

### Step d:

### 2-chloro-6-(2-furyl)-5-[2-(methylthio)pyrimidin-4-yl]nicotinonitrile (Intermediate 12)

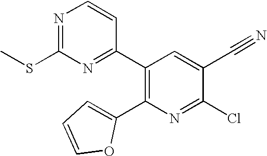

A suspension of 6-(2-furyl)-5-[2-(methylthio)pyrimidin-4-yl]-2-oxo-1,2-dihydropyridine-3-carbonitrile (4.00 g, 12.9 mmol) in phosphorus oxychloride (20 mL) was heated at 110° C. in a sealed vessel and stirred overnight. The mixture was evaporated and carefully neutralised with 4% aqueous sodium hydrogen carbonate solution. Ethyl acetate was added to the solution and, after stirring for five minutes, the mixture was filtered to remove an insoluble black solid. The organic layer was dried over MgSO₄, filtered and evaporated in vacuo to give the title compound (Intermediate 12) (3.71 g, 88%) as a brown solid.

δ ¹H NMR (DMSO): 8.76 (d, 1H), 7.64 (s, 1H), 7.80 (dd, 1H), 7.35 (d, 1H), 7.13 (dd, 1H), 6.68 (dd, 1H), 2.43 (s, 3H).

ESI/MS (m/e, %): 329 [(M+1)⁺, 100].

### Intermediate 13

### 6-(2-furyl)-5-[2-(methylsulfonyl)pyrimidin-4-yl]-1H-pyrazolo[3,4-b]pyridine

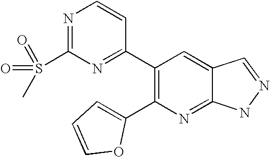

A solution of 6-(2-furyl)-5-[2-(methylthio)pyrimidin-4-yl]-1H-pyrazolo[3,4-b]pyridine (Example 32) (1.00 g, 3.2 mmol) in dichloromethane (33 mL) and methanol (1 mL) was cooled to 0° C. and m-chloroperbenzoic acid (77%, 1.48 g, 6.6 mmol) was added in portions. The mixture was stirred at 0° C. for 10 hours then warmed slowly to room temperature and stirred overnight. The mixture was partitioned between dichloromethane and 4% aqueous sodium hydrogen carbonate solution. The aqueous solution was further extracted with chloroform. The combined organic layers were dried (MgSO₄), evaporated and the residue purified by flash chromatography (98:2 dichloromethane/methanol) to give the title compound (Intermediate 13) (0.48 g, 44%) as a white solid.

δ ¹H NMR (DMSO): 9.07 (d, 1H), 8.59 (s, 1H), 8.30 (s, 1H), 7.79 (d, 1H), 7.60 (dd, 1H), 6.96 (dd, 1H), 6.61 (dd, 1H), 3.28 (s, 3H).

ESI/MS (m/e, %): 342 [(M+1)⁺, 100].

### Intermediate 14

### Step a:

### 6-(2-furyl)-2-oxo-5-pyrimidin-4-yl-1,2-dihydropyridine-3-carboxamide

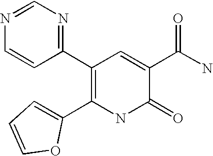

To a mixture 6-(2-furyl)-2-oxo-5-pyrimidin-4-yl-1,2-dihydropyridine-3-carbonitrile (1.0 g, 3.8 mmol) in ethanol (8 mL), water (8.75 mL) and 6M aqueous sodium hydroxide solution (6.25 mL, 37.5 mmol) was added a 30% aqueous hydrogen peroxide solution (2.42 mL, 21.4 mmol). The mixture was heated to 50° C. and stirred overnight. The mixture was cooled and acidified to pH 4-5 using 5M aqueous hydrochloric acid. The precipitate was filtered, washed with water and dried in vacuo to give the title compound (0.86 g, 80%) as a yellow solid, which was used directly without further purification.

ESI/MS (m/e, %): 283 [(M+1)⁺, 100].

### Step b:

### 6-(2-furyl)-2-oxo-5-pyrimidin-4-yl-1,2-dihydropyridine-3-carboxylic acid

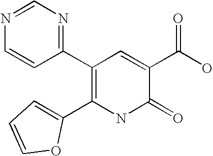

A suspension of 6-(2-furyl)-2-oxo-5-pyrimidin-4-yl-1,2-dihydropyridine-3-carboxamide (0.86 g, 3.04 mmol) in 10% aqueous potassium hydroxide solution (15.2 mL, 27.4 mmol) was heated to 120° C. and stirred overnight. The mixture was cooled and acidified to pH 4-5 using concentrated aqueous hydrochloric acid. The precipitate was filtered, washed with water and dried in vacuo to give the title compound (0.86 g, 100%) as a white solid.

δ ¹H NMR (DMSO): 6.62 (dd, 1H), 7.01 (dd, 1H), 7.20 (dd, 1H), 7.65 (dd, 1H), 8.30 (s, 1H), 8.70 (d, 1H), 9.18 (d, 1H).

ESI/MS (m/e, %): 284 [(M+1)⁺, 100].

### Step c:

### Methyl 2-chloro-6-(2-furyl)-5-pyrimidin-4-ylnicotinate (Intermediate 14)

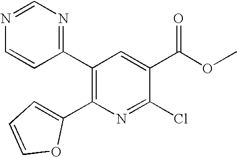

A suspension of 6-(2-furyl)-2-oxo-5-pyrimidin-4-yl-1,2-dihydropyridine-3-carboxylic acid (10.3 g, 36.4 mmol) in phosphorous oxychloride (57 mL) was heated in a sealed tube to 120° C. and stirred overnight. The mixture was cooled and then evaporated to dryness. The resultant oil was cooled in an ice-bath and methanol (120 mL) was slowly added. The mixture was then warmed to room temperature and stirred overnight. The solvent was evaporated and ethyl acetate and water were added. The mixture was then neutralised with solid sodium hydrogen carbonate. A dark insoluble solid was filtered off and discarded. The organic layer was separated, dried (MgSO₄) and evaporated. The residue was triturated with cold diethyl ether and the solid was filtered and dried to give the title compound (Intermediate 14) (7.55 g, 66%) as an off-white solid.

δ ¹H NMR (CDCl₃): 6.50 (dd, 1H), 7.05 (dd, 1H), 7.30 (m, 2H), 8.40 (s, 1H), 8.78 (d, 1H), 9.35 (d, 1H).

ESI/MS (m/e, %): 316 [(M+1)⁺, 100].

### Intermediate 15

### Step a:

### 1-(3-fluorophenyl)-2-pyrimidin-4-ylethanone

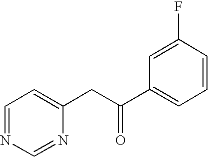

Lithium bis(trimethylsilyl)amide (1.0 M in hexanes, 318.7 mL) was added dropwise over 3 hours to a solution of 4-methylpyrimidine (15 g, 159.3 mmol) and ethyl 3-fluorobenzoate (25.9 mL, 175.3 mmol) in tetrahydrofuran (70 mL) under an atmosphere of nitrogen. The mixture was stirred at ambient temperature for two hours and the precipitate was filtered. The solid was treated with saturated aqueous ammonium chloride solution, filtered, washed with water and dried in vacuo to give the title compound (32.4 g, 99%) as a yellow solid.

### Step b:

### (2E)-3-(dimethylamino)-1-(3-fluorophenyl)-2-pyrimidin-4-ylprop-2-en-1-one

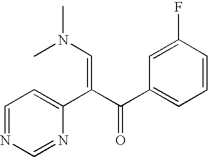

A suspension of 1-(3-fluorophenyl)-2-pyrimidin-4-ylethanone (32.4 g, 158.7 mmol) in N,N-dimethylformamide dimethyl acetal (85 mL) was heated to reflux. The mixture was stirred for 5 hours and then evaporated to give the title compound as a dark oil (39.2 g, 91%).

### Step c:

### 6-(3-fluorophenyl)-2-oxo-5-pyrimidin-4-yl-1,2-dihydropyridine-3-carbonitrile

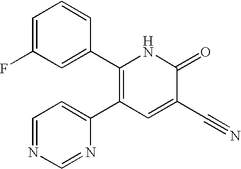

Sodium methoxide (18.76 g, 347.3 mmol) was added to a mixture of (2E)-3-(dimethylamino)-1-(3-fluorophenyl)-2-pyrimidin-4-ylprop-2-en-1-one (39.2 g, 144.7 mmol) and 2-cyanoacetamide (14.59 μg, 173.5 mmol) in N,N-dimethylformamide (300 mL) under an atmosphere of argon. The mixture was heated to 80° C. and stirred for six hours, then concentrated under high vacuum at 65° C. Water was added to the residue and the pH adjusted to 4-5 with 5M aqueous hydrochloric acid. The precipitate was filtered and dried in vacuo to give the title compound (36.5 g, 86%) as a red solid.

δ ¹H NMR (DMSO): 6.97 (dd, 1H), 7.09 (d, 1H), 7.49-7.28 (m, 3H), 8.54 (s, 1H), 8.57 (d, 1H), 9.08 (d, 1H).

### Step d:

### 2-chloro-6-(3-fluorophenyl)-5-pyrimidin-4-ylnicotinonitrile (Intermediate 15)

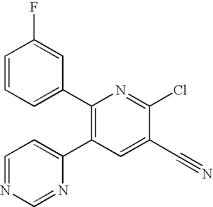

A suspension of 6-(3-fluorophenyl)-2-oxo-5-pyrimidin-4-yl-1,2-dihydropyridine-3-carbonitrile (27.84 g, 95.2 mmol) in phosphorus oxychloride (40 mL) was heated to reflux and stirred for 16 hours. The mixture was evaporated, ice-water was added and the mixture was neutralised with aqueous ammonia. The solid was filtered and washed thoroughly with dichloromethane, and the organic phase of the filtrate was dried and evaporated. The solid residue was filtered through silica gel washing with dichloromethane to yield the title compound (Intermediate 15) (18.35 g, 62%) as orange crystals.

### Intermediate 16

### Step a:

### 2-[(2,4-dimethoxybenzyl)amino]-6-(2-furyl)-6-pyrimidin-4-ylnicotinonitrile

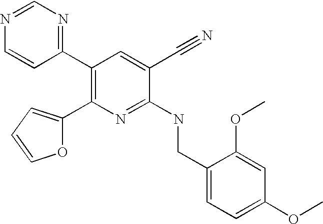

A mixture of 2-chloro-6-(2-furyl)-5-pyrimidin-4-ylnicotinonitrile (0.99 g, 3.50 mmol), 3,4-dimethoxybenzylamine (1.19 g, 7.12 mmol) and triethylamine (0.385 g, 3.80 mmol) in ethanol (13 mL) was heated at 175° C. for 50 minutes in Biotage Initiator Microwave Synthesizer. The mixture was then poured into water and extracted with ethyl acetate. The organic layer was dried (MgSO₄) and evaporated. The resultant oil was taken up in dichloromethane (45 mL), polymer supported benzaldehyde resin (4.82 g, 6.03 mmol of active aldehyde residues) was added and the mixture was shaken overnight. The mixture was filtered and the resin was washed with tetrahydrofuran. The combined filtrate and washings were evaporated to give the title compound (1.50 g, 100%) as an oil.

δ ¹H NMR (CDCl₃): 9.25 (d, 1H), 8.60 (d, 1H), 7.98 (s, 1H), 7.30 (m, 2H), 7.10 (m, 2H), 6.50 (m, 3H), 6.00 (t, 1H), 4.75 (d, 2H), 3.90 (s, 3H), 3.85 (s, 3H).

ESI/MS (m/e, %): 414 [(M+1)⁺, 100].

### Step b:

### 2-[(2,4-dimethoxybenzyl)amino]-6-(2-furyl)-5-pyrimidin-4-ylnicotinic Acid

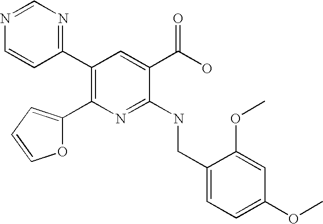

A suspension of 2-[(2,4-dimethoxybenzyl)amino]-6-(2-furyl)-5-pyrimidin-4-ylnicotinonitrile (3.5 mmol) and potassium hydroxide (0.85 g, 15.2 mmol) in ethylene glycol (18 mL) was heated to 150° C. After stirring for four hours the yellow solution was cooled, poured onto ice water and taken to pH 5 with 5M aqueous hydrochloric acid. The precipitate was filtered, washed with water and dried in vacuo to give the title compound (1.40 g, 93%) as a white solid.

δ ¹H NMR (DMSO): 9.25 (d, 1H), 8.75 (s, 1H), 8.60 (d, 1H), 8.25 (s, 1H), 7.60 (d, 1H), 7.25 (m, 2H), 7.00 (d, 1H), 6.40-6.70 (m, 3H), 4.70 (d, 2H), 3.80 (s, 3H), 3.65 (s, 3H).

ESI/MS (m/e, %): 433 [(M+1)⁺, 100].

### Step c:

### 3-(2,4-Dimethoxy-benzyl)-5-furan-2-yl-6-pyrimidin-4-yl-1,3-dihydro-imidazo[4,5-b]pyridin-2-one

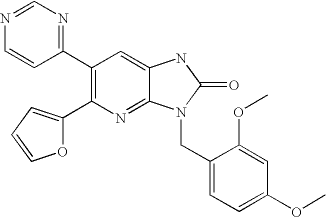

Diphenylphosphoryl azide (0.76 g, 2.77 mmol) was added to a mixture of 2-[(2,4-dimethoxybenzyl)amino]-6-(2-furyl)-5-pyrimidin-4-ylnicotinic acid (1.00 g, 2.31 mmol) and triethylamine (0.47 g, 4.63 mmol) in 1,4-dioxane (20 mL). The mixture was heated to reflux, stirred for 6 hours and then cooled. The solvent was evaporated, water was added and the mixture extracted with ethyl acetate. The organic layer was washed with 4% aqueous sodium hydrogen carbonate solution, brine and dried (MgSO₄). The solvent was evaporated and the residue triturated with diethyl ether to give the title compound (0.860 g, 87%) as a yellow solid.

δ ¹H NMR (DMSO): 11.50 (s, 1H), 9.20 (d, 1H), 8.70 (d, 1H), 7.50 (d, 1H), 7.45 (s, 1H), 7.25 (dd, 1H), 6.90 (d, 1H), 6.35-6.60 (m, 4H), 4.95 (s, 2H), 3.80 (s, 3H), 3.70 (s, 3H).

ESI/MS (m/e, %): 430 [(M+1)⁺, 100].

### Step d:

### 3-(2,4-Dimethoxy-benzyl)-5-furan-2-yl-1-methyl-6-pyrimidin-4-yl-1,3-dihydro-imidazo[4,5-b]pyridin-2-one (Intermediate 16)

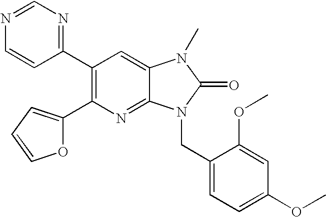

To a solution of 3-(2,4-Dimethoxy-benzyl)-5-furan-2-yl-6-pyrimidin-4-yl-1,3-dihydro-imidazo[4,5-b]pyridin-2-one (0.30 g, 0.70 mmol) in N,N-dimethylformamide (3 mL) was added portionwise 60% sodium hydride in mineral oil (0.056 g, 1.4 mmol). After hydrogen evolution had ceased, methyl iodide (0.119 g, 0.84 mmol) was added and the mixture was stirred overnight. The mixture was partitioned between ethyl acetate and water and the organic layer was washed with water and brine, dried (MgSO₄) and evaporated to give the title compound (Intermediate 16) (0.263 g, 85%) as a white solid.

δ ¹H NMR (CDCl₃): 9.25 (d, 1H), 8.60 (d, 1H), 7.45 (s, 1H), 7.25 (m, 2H), 7.02 (dd, 1H), 6.80 (dd, 1H), 6.40-6.50 (m, 4H), 5.20 (s, 2H), 3.82 (s, 3H), 3.77 (s, 3H), 3.43 (s, 3H).

ESI/MS (m/e, %): 444 [(M+1)⁺, 100].

### Intermediate 17

### Step a:

### 6-(2-furyl)-5-pyrimidin-4-ylpyridin-2-amine

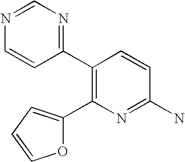

A mixture of 2-amino-6-(2-furyl)-5-pyrimidin-4-ylnicotinic acid (0.50 g, 1.8 mmol), quinoline (5 mL) and copper powder (0.09 g) was heated at 230° C. for 60 minutes in a Biotage Initiator Microwave Synthesiser. The mixture was diluted with diethylether and purified by flash chromatography (diethylether followed by ethyl acetate) to give the title compound (0.30 g, 70%) as a yellow solid.

δ ¹H NMR (CDCl₃): 4.80 (s, 2H), 6.42 (dd, 1H), 6.55 (d, 1H), 6.63 (dd, 1H), 7.00 (dd, 1H), 7.29 (d, 1H), 7.80 (d, 1H), 8.59 (d, 1H), 9.20 (d, 1H).

ESI/MS (m/e, %): 239 [(M+1)⁺, 100].

### Step b:

### 3-bromo-6-(2-furyl)-5-pyrimidin-4-ylpyridin-2-amine (Intermediate 17)

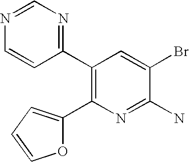

To a solution of 6-(2-furyl)-5-pyrimidin-4-ylpyridin-2-amine (5.0 g, 21.0 mmol) in dimethylsulfoxide (27 mL) and water (27 mL) was added N-bromosuccinimide (3.74 g, 21.0 mmol) portionwise over 20 minutes. After 20 minutes, more N-bromosuccinimide (0.50 g, 2.8 mmol) was added and the mixture was stirred for a further 20 minutes. The mixture was diluted with water and extracted with ethyl acetate. The combined organic layers were washed with water and brine, dried (MgSO₄) and concentrated in vacuo. The residue was purified by flash chromatography (1:1 hexanes/ethyl acetate) to give the title compound (Intermediate 17) (2.30 g, 35%) as a white solid.

δ ¹H NMR (CDCl₃): 9.26 (d, 1H), 8.60 (d, 1H), 8.05 (s, 1H), 7.35 (m, 1H), 7.04 (dd, 1H), 6.69 (dd, 1H), 6.45 (m, 1H), 5.38 (s, 2H)

ESI/MS (m/e, %): 317/319 [(M+1)⁺, 100]

### Intermediate 18

### 3-ethynyl-6-(2-furyl)-5-pyrimidin-4-ylpyridin-2-amine

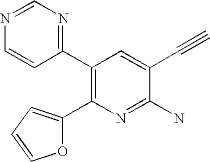

To a solution of 3-bromo-6-(2-furyl)-5-pyrimidin-4-ylpyridin-2-amine (Intermediate 17) (1.30 g, 4.10 mmol) in tetrahydrofuran (4 mL) under an atmosphere of argon was added triethylamine (6 mL), copper(I) iodide (0.039 g, 0.205 mmol), bis(triphenylphosphine)palladium(II) chloride (0.144 g, 0.205 mmol) and trimethylsilylacetylene (0.805 g, 8.2 mmol). The mixture was heated to 90° C. in a sealed tube and stirred overnight. The mixture was cooled, diluted with water and extracted with ethyl acetate. The organic layer was dried (MgSO₄) and the residue was purified by flash chromatography (1:1 hexanes/ethyl acetate) to give 6-furan-2-yl-5-pyrimidin-4-yl-3-trimethylsilanylethynyl-pyridin-2-ylamine (0.645 g, 47%) as a white solid. This material was dissolved in methanol (20 mL) and potassium carbonate (0.263 g, 1.9 mmol) was added. After stirring for 2 hours, water was added and the mixture was extracted with dichloromethane. The organic layer was washed with water, dried (MgSO₄) and evaporated to give the title compound (Intermediate 18) (0.500 g, 100%) as a yellow solid.

δ ¹H NMR (CDCl₃): 9.25 (d, 1H), 8.60 (d, 1H), 7.91 (s, 1H), 7.33 (m, 1H), 7.09 (dd, 1H), 6.71 (dd, 1H), 6.45 (m, 1H), 5.53 (s, 2H), 3.47 (s, 1H).

ESI/MS (m/e, %): 263 [(M+1)⁺, 100].

### Intermediate 19

### 6-(2-furyl)-3-(phenylethynyl)-5-pyrimidin-4-ylpyridin-2-amine

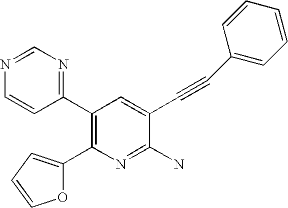

To a solution of 3-bromo-6-(2-furyl)-5-pyrimidin-4-ylpyridin-2-amine (Intermediate 17) (0.20 g, 0.63 mmol) in tetrahydrofuran (2 mL) under an atmosphere of argon was added triethylamine (3 mL), copper(I) iodide (0.005 g, 0.025 mmol), bis(triphenylphosphine)palladium(II) chloride (0.018 g, 0.025 mmol) and ethynylbenzene (0.13 g, 1.26 mmol). The mixture was heated to 90° C. in a sealed tube and stirred overnight. The mixture was cooled, diluted with water and extracted with ethyl acetate. The organic layer was dried (MgSO₄) and the residue was purified by flash chromatography (1:1 hexanes/ethyl acetate) to give the title compound (Intermediate 19) (0.054 g, 25%) as a yellow solid.

δ ¹H NMR (CDCl₃): 9.25 (d, 1H), 8.62 (d, 1H), 7.95 (s, 1H), 7.35-7.05 (m, 7H), 6.72 (dd, 1H), 6.45 (m, 1H), 5.40 (s, 2H).

ESI/MS (m/e, %): 339 [(M+1)⁺, 100].

### Intermediate 20

### 3-[(3-fluorophenyl)ethynyl]-6-(2-furyl)-5-pyrimidin-4-ylpyridin-2-amine

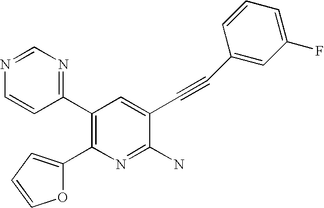

To a solution of 3-bromo-6-(2-furyl)-5-pyrimidin-4-ylpyridin-2-amine (Intermediate 17) (0.420 g, 1.3 mmol) in tetrahydrofuran (3 mL) under an atmosphere of argon was added triethylamine (5 mL), copper(I) iodide (0.010 g, 0.05 mmol), bis(triphenylphosphine)palladium(II) chloride (0.036 g, 0.05 mmol) and 1-ethynyl-3-fluorobenzene (0.312 g, 2.6 mmol). The mixture was heated to 90° C. in a sealed tube and stirred overnight. The mixture was cooled, diluted with water and extracted with ethyl acetate. The organic layer was dried (MgSO₄) and the residue was purified by flash chromatography (1:1 hexanes/ethyl acetate) to give the title compound (Intermediate 20) (0.185 g, 40%) as a yellow solid.

δ ¹H NMR (CDCl₃): 9.26 (d, 1H), 8.62 (d, 1H), 7.95 (s, 1H), 7.34-7.08 (m, 6H), 6.72 (dd, 1H), 6.45 (m, 1H), 5.39 (s, 2H).

ESI/MS (m/e, %): 357 [(M+1)⁺, 100].

### Intermediate 21

### Step a:

### 6-(2-furyl)-1-(4-methoxybenzyl)-5-pyrimidin-4-yl-1,2-dihydro-3H-pyrazolo[3,4-b]pyridin-3-one

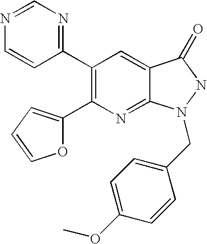

To methyl 2-chloro-6-(2-furyl)-5-pyrimidin-4-ylnicotinate (Intermediate 14) (0.30 g, 0.95 mmol) in ethanol (5 mL) was added (4-methoxybenzyl)hydrazine (0.69 g, 5.7 mmol) and the mixture was heated to 65° C. in a sealed tube and stirred overnight. The mixture was concentrated to dryness and the residue purified by flash chromatography (9:1 dichloromethane/methanol) to give the title compound (0.24 g, 63%) as an off-white solid.

δ ¹H NMR (CDCl₃): 9.26 (d, 1H), 8.65 (d, 1H), 8.30 (s, 1H), 7.39-7.20 (m, 4H), 7.04 (dd, 1H), 6.85 (d, 2H), 6.55 (m, 1H), 5.47 (s, 2H), 3.90 (s, 3H).

ESI/MS (m/e, %): 400 [(M+1)⁺, 100].

### Step b:

### 3-Ethoxy-6-furan-2-yl-1-(4-methoxybenzyl)-5-pyrimidin-4-yl-1H-pyrazolo[3,4-b]pyridine (Intermediate 21)

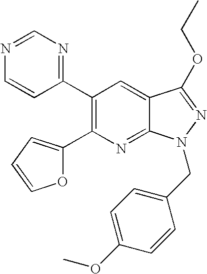

To a solution of 6-(2-furyl)-1-(4-methoxybenzyl)-5-pyrimidin-4-yl-1,2-dihydro-3H-pyrazolo[3,4-b]pyridin-3-one (0.100 g, 0.25 mmol) in N,N-dimethylformamide (1.5 mL) was added portionwise 60% sodium hydride in mineral oil (0.012 mg, 0.3 mmol). After hydrogen evolution had ceased, ethyl bromide (0.033 g, 0.3 mmol) was added and the mixture was stirred for a further 30 minutes. The mixture was partitioned between ethyl acetate and water and the organic layer was washed with brine, dried (MgSO₄) and evaporated. Flash chromatography (1:1 hexanes/ethyl acetate) gave the title compound (Intermediate 21) (0.054 g, 50%) as a yellow solid.

δ ¹H NMR (CDCl₃): 9.25 (d, 1H), 8.63 (d, 1H), 8.25 (s, 1H), 7.38-7.30 (m, 3H), 7.18 (dd, 1H), 6.96 (dd, 1H), 6.82 (d, 2H), 6.55 (m, 1H), 5.5 (s, 2H), 4.10 (m, 2H), 3.80 (s, 3H), 1.45 (m, 3H).

ESI/MS (m/e, %): 428 [(M+1)⁺, 100].

### Intermediate 22

### 6-(2-Furyl)-5-pyrimidin-4-yl-1H-pyrazolo[3,4-b]pyridin-3-amine

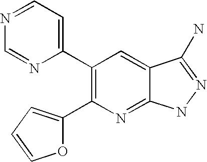

To a suspension of 2-chloro-6-(2-furyl)-5-pyrimidin-4-ylnicotinonitrile (1.14 g, 4.0 mmol) in ethyl alcohol (20 mL) was added hydrazine monohydrate (0.61 g, 12.1 mmol) and the mixture was heated to reflux and stirred overnight. The mixture was treated with 4% aqueous sodium hydrogen carbonate solution and the precipitate was filtered, washed with water, ethyl acetate and ethanol and dried in vacuo to give the title compound (Intermediate 22) (0.80 g, 71%) as an orange solid.

δ ¹H NMR (DMSO): 5.83 (s, 2H), 6.58 (dd, 1H), 6.80 (dd, 1H), 7.25 (dd, 1H), 7.60 (dd, 1H), 8.42 (s, 1H), 8.74 (d, 1H), 9.20 (d, 1H), 12.25 (s, 1H).

ESI/MS (m/e, %): 279 [(M+1)⁺, 100].

### EXAMPLES

### Example 1

### 2-(3-Fluorophenyl)-3,4′-bipyridine-5,6-diamine

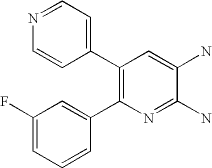

A suspension of 2-(3-fluorophenyl)-5-nitro-3,4′-bipyridin-6-amine (intermediate 2) (0.15 g, 0.5 mmol) and 10% palladium on carbon (30 mg) in ethanol (10 mL) was stirred under an atmosphere of hydrogen. After 1 hour, the mixture was filtered through Celite® and the filter cake was washed with ethanol. The combined filtrate and washings were evaporated to give the title compound (0.14 g, 100%) as a pale brown solid.

δ ¹H NMR (CDCl₃): 3.50 (s, 2H), 4.50 (s, 2H), 6.9-7.20 (m, 7H), 8.45 (d, 2H).

ESI/MS (m/e, %): 281 [(M+1)⁺, 100].

### Example 2

### 5-(3-Fluorophenyl)-6-pyridin-4-yl-3H-imidazo[4,5-b]pyridine

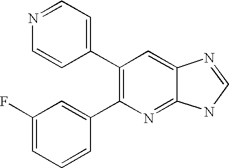

A mixture of 2-(3-fluorophenyl)-3,4′-bipyridine-5,6-diamine (Example 1) (65 mg, 0.23 mmol) and triethylorthoformate (68 mg, 0.46 mmol) in glacial acetic acid (0.15 mL) was heated in a sealed tube to 140° C. After stirring overnight, the mixture was cooled and taken to pH 7 with saturated aqueous sodium hydrogen carbonate solution and then extracted. The organic layer was dried (MgSO₄) and evaporated to give a solid which was triturated with diethyl ether and dried to give the title compound (36 mg, 54%) as an off-white solid.

δ ¹H NMR (DMSO): 7.00-7.40 (m, 6H), 8.10 (m, 1H), 8.47 (d, 2H), 8.57 (s, 1H).

ESI/MS (m/e, %): 291 [(M+1)⁺, 100].

### Example 3

### 5-(3-Fluorophenyl)-2-methyl-6-pyridin-4-yl-3H-imidazo[4,5-b]pyridine

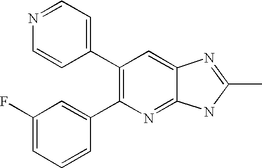

A mixture of 2-(3-fluorophenyl)-3,4′-bipyridine-5,6-diamine (Example 1) (42 mg, 0.15 mmol) and triethylorthoacetate (49 mg, 0.3 mmol) in glacial acetic acid (0.15 mL) was heated in a sealed tube to 140° C. After stirring overnight, the mixture was cooled and taken to pH 7 with saturated aqueous sodium hydrogen carbonate solution and then extracted. The organic layer was dried (MgSO₄) and evaporated to give a solid which was triturated with diethyl ether and dried to give the title compound (29 mg, 63%) as an off-white solid.

δ ¹H NMR (DMSO): 6.96-7.38 (m, 6H), 7.93 (s, 1H), 8.40 (d, 2H).

ESI/MS (m/e, %): 305 [(M+1)⁺, 100].

### Example 4

### 2-Cyclopropyl-5-(3-fluorophenyl)-6-pyridin-4-yl-3H-imidazo[4,5-b]pyridine

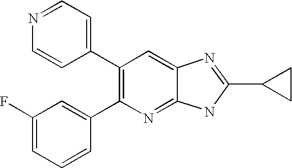

N-[6-amino-2-(3-fluorophenyl)-3,4′-bipyridin-5-yl]cyclopropanecarboxamide (Intermediate 6) (35 mg, 0.1 mmol) in glacial acetic acid (2 mL) was heated in a sealed tube to 140° C. After stirring for two days, the mixture was cooled and evaporated. Flash chromatography (98:2 dichloromethane/methanol) gave the title compound (16 mg, 48%) as a white solid.

δ ¹H NMR (DMSO): 0.85 (m, 2H), 0.99 (m, 1H), 1.21 (m, 2H), 7.0-7.20 (m, 6H), 7.94 (s, 1H), 8.52 (d, 2H), 11.42 (s, 1H).

ESI/MS (m/e, %): 331 [(M+1)⁺, 100].

### Example 5

### 2-Ethyl-5-(3-fluorophenyl)-6-pyridin-4-yl-3H-imidazo[4,5-b]pyridine

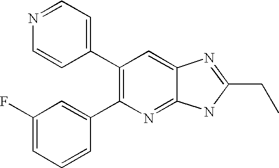

A mixture of 2-(3-fluorophenyl)-3,4′-bipyridine-5,6-diamine (Example 1) (70 mg, 0.25 mmol) and triethylorthopropionate (88 mg, 0.50 mmol) in glacial acetic acid (3 mL) was heated in a sealed tube to 140° C. After stirring four hours, the mixture was cooled and evaporated. The mixture was taken up in a small amount of water and taken to pH 7 with saturated aqueous sodium hydrogen carbonate solution and then extracted. The organic layer was dried (MgSO₄) and evaporated to give a solid which was purified by flash chromatography (98:2 dichloromethane/methanol) give the title compound (22 mg, 30%) as a white solid.

δ ¹H NMR (DMSO): 1.20 (t, 3H), 2.39 (q, 2H), 7.0-7.35 (m, 6H), 8.03 (s, 1H), 8.50 (d, 2H), 12.70 (s, 1H).

ESI/MS (m/e, %): 319 [(M+1)⁺, 100].

### Example 6

### 5-(3-Fluorophenyl)-6-pyridin-4-yl-3H-[1,2,3]triazolo[4,5-b]pyridine

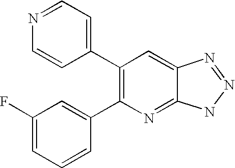

A solution of sodium nitrite (20 mg, 0.29 mmol) in water (3 mL) was added dropwise to a cooled (ice-bath) solution of 2-(3-fluorophenyl)-3,4′-bipyridine-5,6-diamine (Example 1) (69 mg, 0.25 mmol) in glacial acetic acid (2 mL) and water (1.0 mL). The mixture was stirred 30 minutes and then warmed to room temperature and stirred overnight. Solid sodium hydrogen carbonate was added in small portions. The precipitate was filtered, washed with water and dried to give the title compound (42 mg, 59%) as an off-white solid.

δ ¹H NMR (DMSO): 7.00-7.40 (m, 6H), 8.55 (d, 2H), 8.60 (s, 1H).

ESI/MS (m/e, %): 292 [(M+1)⁺, 100].

### Example 7

### 5-(3-Fluorophenyl)-6-pyridin-4-yl-1,3-dihydro-2H-imidazo[4,5-b]pyridin-2-one

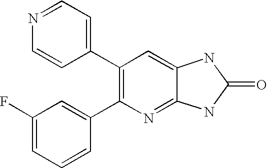

To a warm (40° C.) solution of 2-(3-fluorophenyl)-3,4′-bipyridine-5,6-diamine (Example 1) (64.6 mg, 0.23 mmol) in dimethylformamide (0.4 mL) was added N,N′-carbonyldiimidazole (40.4 mg, 0.25 mmol). The mixture was stirred at room temperature for two hours then warmed- to 80° C. After stirring five hours, the mixture was poured into water and the precipitate that formed was filtered and washed with water and diethyl ether to give the title compound (41 mg, 59%) as an off-white solid.

δ ¹H NMR (DMSO): 6.95-7.3 (m, 7H), 8.45 (d, 2H).

ESI/MS (m/e, %): 307 [(M+1)⁺, 100].

### Examples 8 and 9

### 5-Ethynyl-2-(3-fluorophenyl)-3,4′-bipyridin-6-amine

### 6-(3-Fluorophenyl)-5-pyridin-4-yl-1H-pyrrolo[2,3-b]pyridine

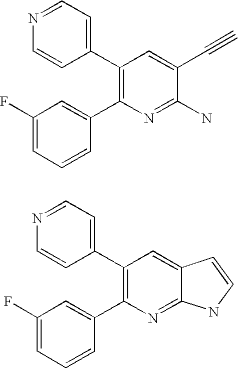

To a solution 2-(3-fluorophenyl)-5-[(trimethylsilyl)ethynyl]-3,4′-bipyridin-6-amine (intermediate 3) (0.29 mmol) in dry N,N-dimethylformamide (3.5 mL) under an atmosphere of argon was added copper(I) iodide (2.2 mg, 0.012 mmol) and the mixture was heated to reflux. After stirring overnight, the mixture was cooled, diluted with water and extracted with ethyl acetate. The organic layer was dried (MgSO₄), evaporated and the residue purified by flash chromatography (200:1 dichloromethane/methanol to 50:1 dichloromethane/methanol) to give 5-ethynyl-2-(3-fluorophenyl)-3,4′-bipyridin-6-amine (13.6 mg, 16%) as a white solid: δ ¹H NMR (CDCl₃): 3.50 (s, 1H), 5.25 (s, 2H), 6.8-7.23 (m, 6H), 7.63 (s, 1H), 8.45 (m, 2H). ESI/MS (m/e, %): 290 [(M+1)⁺, 100] and 6-(3-fluorophenyl)-5-pyridin-4-yl-1H-pyrrolo[2,3-b]pyridine (6.5 mg, 8%) as a white solid: δ ¹H NMR (CDCl₃): 6.55 (m, 1H), 7.0-7.4 (m, 6H), 7.53 (m, 1H), 7.64 (m, 1H), 8.00 (s, 1H), 11.0 (s, 1H). ESI/MS (m/e, %): 290 [(M+1)⁺, 100].

### Example 10

### 6-(2-Furyl)-5-pyrimidin-4-yl-1H-pyrazolo[3,4-b]pyridin-3-amine

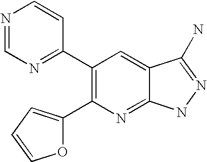

To a suspension of 2-chloro-6-(2-furyl)-5-pyrimidin-4-ylnicotinonitrile (Intermediate 4) (1.14 g, 4.0 mmol) in ethyl alcohol (20 mL) was added hydrazine monohydrate (0.61 g, 12.1 mmol) and the mixture was heated to reflux and stirred overnight. The mixture was treated with 4% aqueous sodium hydrogen carbonate solution and the precipitate was filtered, washed with water, ethyl acetate and ethanol and dried in vacuo to give the title compound (0.80 g, 71%) as an orange solid.

δ ¹H NMR (DMSO): 5.83 (s, 2H), 6.58 (dd, 1H), 6.80 (dd, 1H), 7.25 (dd, 1H), 7.60 (dd, 1H), 8.42 (s, 1H), 8.74 (d, 1H), 9.20 (d, 1H), 12.25 (s, 1H).

ESI/MS (m/e, %): 279 [(M+1)⁺, 100].

### Example 11

### N-[6-(2-furyl)-5-pyrimidin-4-yl-1H-pyrazolo[3,4-b]pyridin-3-yl]acetamide

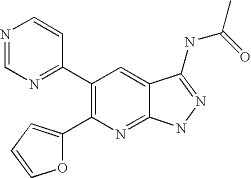

To a suspension of 6-(2-furyl)-5-pyrimidin-4-yl-1H-pyrazolo[3,4-b]pyridin-3-amine (Example 10) (0.101 g, 0.36 mmol) in pyridine (0.5 mL) was added acetic anhydride (0.038 mL, 0.4 mmol) and the mixture was heated to reflux. After 20 hours the mixture was cooled and poured into water. The precipitate was filtered, washed with water and dried in the air to give the title compound (0.084 g, 72%) as an orange solid.

δ ¹H NMR (DMSO): 2.10 (s, 3H), 6.59 (dd, 1H), 6.79 (dd, 1H), 7.38 (dd, 1H), 7.61 (dd, 1H), 8.60 (s, 1H), 8.78 (d, 1H), 9.22 (d, 1H), 10.86 (s, 1H), 13.46 (s, 1H).

ESI/MS (m/e, %): 321 [(M+1)⁺, 100].

### Example 12

### 5-(2-Furyl)-6-pyrimidin-4-yl-1,3-dihydro-2H-imidazo[4,5-b]pyridin-2-one

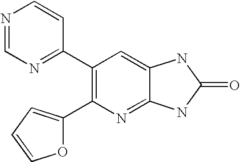

Triethylamine (0.10 mL, 0.72 mmol) was added to a mixture of 2-amino-6-(2-furyl)-5-pyrimidin-4-ylnicotinic acid (Intermediate 5) (0.10 g, 0.35 mmol) and diphenylphosphoryl azide (0.127 g, 0.46 mmol) in 1,4-dioxane (2 mL). The mixture was heated to reflux and stirred overnight. The mixture was evaporated and to the residue was added glacial acetic acid (0.13 mL) and water and after scratching the precipitate was filtered, washed with water and methanol and dried to give the title compound (0.055 g, 56%) as a yellow solid.

δ ¹H NMR (DMSO): 6.51 (dd, 1H), 6.59 (dd, 1H), 7.23 (dd, 1H), 7.38 (s, 1H), 7.50 (m, 1H), 8.71 (d, 1H); 9.19 (d, 1H) 11.16 (s, 1H), 11.69 (s, 1H).

ESI/MS (m/e, %): 280 [(M+1)⁺, 100].

### Example 13

### 2-(2-thienyl)-3,4′-bipyridine-5,6-diamine

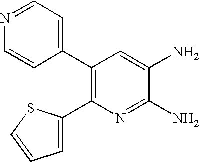

A suspension of 5-nitro-2-(2-thienyl)-3,4′-bipyridin-6-amine (Intermediate 7) (91.5 mg, 0.31 mmol) and 10% palladium on carbon (9.15 mg) in ethanol (5 mL) was stirred under hydrogen atmosphere. After 1 day, the mixture was filtered through Celite® and the filter cake was washed with ethanol. The combined filtrate and washings were evaporated to give the title compound (47.6 mg, 57%).

δ ¹H NMR (CDCl₃): 6.52 (d, 1H), 679 (m, 2H), 7.24 (m, 3H), 8.57 (d, 2H).

ESI/MS (m/e, %): 269 [(M+1)⁺, 100].

### Example 14

### 2-(2-Furyl)-3,4′-bipyridine-5,6-diamine

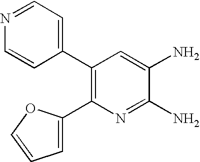

A suspension of 2-(2-furyl)-5-nitro-3,4′-bipyridin-6-amine (Intermediate 9) (114.1 mg, 0.4 mmol) and 10% palladium on carbon (11.4 mg) in ethanol (5 mL) was stirred under an hydrogen atmosphere. After 1 day, the mixture was filtered through Celite® and the filter cake was washed with ethanol. The combined filtrate and washings were evaporated. The residue was purified by flash chromatography (1:1 hexane/ethyl acetate) to give the title compound as a solid (69.6 mg, 68%).

δ ¹H NMR (CDCl₃): 6.10 (d, 1H), 6.29 (q, 1H), 6.83 (s, 1H), 7.17 (dd, 2H), 7.31 (m, 1H), 8.57 (dd, 2H).

ESI/MS (m/e, %): 253 [(M+1)⁺, 100].

### Example 15

### 6-(2-Furyl)-5-[2-(methylthio)pyrimidin-4-yl]pyridine-2,3-diamine

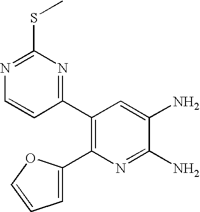

A solution of 6-(2-furyl)-5-[2-(methylthio)pyrimidin-4-yl]-3-nitropyridin-2-amine (intermediate 8) (75 mg, 0.23 mmol), iron powder (56 mg, 1 mmol) and a catalytic amount of hydrogen chloride in ethanol (3 mL) was heated to reflux. After 3 hours, the mixture was evaporated and aqueous sodium hydrogen carbonate solution (4%) was added and then extracted with ethyl acetate. The organic layer was dried and evaporated. The residue was purified by flash chromatography (7:3 hexane/ethyl acetate) to give the title compound as a solid (47 mg, 68%).

δ ¹H NMR (CDCl₃): 6.40 (m, 1H), 6.50 (m, 1H), 6.62 (d, 1H), 7.29 (s, 1H), 7.33 (m, 1H), 8.32 (s, 1H), 8.34 (s, 1H).

ESI/MS (m/e, %): 300 [(M+1)⁺, 100].

### Example 16

### 6-(2-Furyl)-5-pyrimidin-4-ylpyridine-2,3-diamine

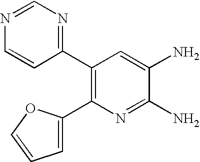

A mixture of 2-chloro-6-(2-furyl)-5-pyrimidin-4-ylpyridin-3-amine (Intermediate 11) (72 mg, 0.364 mmol) and copper (I) chloride (11 mg, 0.113 mmol) in aqueous ammonia (1 mL) was heated in a sealed tube to 120° C. After 1 day, the mixture was filtered and concentrated. The residue was purified by flash chromatography (98:2 dichloromethane/methanol) to give the title compound as a solid (14 mg, 21%).

δ ¹H NMR (DMSO): 5.11 (m, 1H), 6.00 (m, 1H), 6.38-6.46 (m, 2H), 6.86 (d, 1H), 7.02 (s, 1H), 7.42 (m, 1H), 9.08 (m, 1H).

ESI/MS (m/e, %): 254 [(M+1)⁺, 100].

### Example 17

### 6-Pyridin-4-yl-5-(2-thienyl)-1,3-dihydro-2H-imidazo[4,5-b]pyridin-2-one

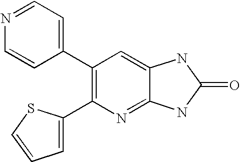

A mixture of 2-(2-thienyl)-3,4′-bipyridine-5,6-diamine (Example 13) (0.048 g, 0.177 mmol) and carbonyl diimidazole (0.032 g, 0.195 mmol) in of dioxane (2 mL) was heated at 100° C. for 48 hours. The solvent was evaporated and the crude mixture was purified by flash chromatography (95:5 dichloromethane/methanol) to give the title compound (0.051 g, 70%).

δ ¹H NMR (DMSO): 6.36-6.68 (m, 2H), 6.80-6.85 (dd, 1H), 7.00 (s, 1H), 7.10 (s, 1H), 7.32.7.35 (m, 2H), 7.44-7.47 (m, 1H), 7.63 (s, 1H), 8.57-8.60 (m, 2H).

ESI/MS (m/e, %): 295 [(M+1)⁺, 100].

### Example 18

### 2-Ethoxy-5-(2-furyl)-6-pyrimidin-4-yl-3H-imidazo[4,5-b]pyridine

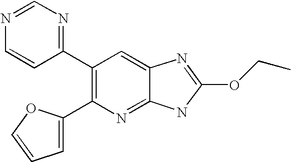

A mixture of 6-(2-furyl)-5-pyrimidin-4-ylpyridine-2,3-diamine (Example 16) (30 mg, 0.118 mmol) and tetraethyl orthocarbonate (46 mg, 0.239 mmol) in glacial acetic acid (1.5 mL) was stirred at room temperature for 5 hours. Then, the mixture was concentrated and 1.5 mL of dioxane and a catalytic amount of glacial acetic acid were added. The mixture was heated to 100° C. and stirred overnight, then it was evaporated and the residue purified by flash chromatography (98:2 dichloromethane/methanol) to give the title compound (20 mg, 55%).

δ ¹H NMR (CDCl₃): 1.46 (t, 3H), 4.13 (q, 2H), 6.47 (m, 2H), 6.78 (d, 1H), 7.05 (d, 1H), 7.65 (s, 1H), 8.63 (d, 1H), 9.30 (s, 1H), 10.59 (s, 1H).

ESI/MS (m/e, %): 308 [(M+1)⁺, 100].

### Example 19

### 5-(2-furyl)-6-pyrimidin-4-yl-3H-imidazo[4,5-b]pyridine

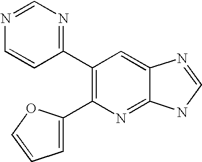

A mixture of 6-(2-furyl)-5-pyrimidin-4-ylpyridine-2,3-diamine (Example 16) (45 mg, 0.178 mmol) and triethylorthoformate (53 mg, 0.355 mmol) in glacial acetic acid (2 mL) was heated in a sealed tube to 140° C. After stirring one hour, the mixture was cooled, taken up in a small amount of water and taken to pH 7 with saturated aqueous sodium hydrogen carbonate solution. The precipitate was filtered, washed with water and dried to give the title compound (20 mg, 43%).

ESI/MS (m/e, %): 264 [(M+1)⁺, 100].

### Example 20

### 5-(2-Furyl)-2-methyl-6-pyrimidin-4-yl-3H-imidazo[4,5-b]pyridine

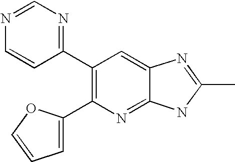

A mixture of 6-(2-furyl)-5-pyrimidin-4-ylpyridine-2,3-diamine (Example 16) (45 mg, 0.178 mmol) and triethylorthoacetate (58 mg, 0.355 mmol) in glacial acetic acid (2 mL) was heated in a sealed tube to 140° C. After stirring one hour, the mixture was cooled and taken up in a small amount of water and taken to pH 7 with saturated aqueous sodium hydrogen carbonate solution and then extracted with ethyl acetate. The organic layer was dried and evaporated to give the title compound (40 mg, 81%) as a solid.

δ ¹H NMR (CDCl₃): 2.45 (s, 3H), 6.49 (m, 1H), 7.22 (m, 2H), 7.40 (s, 1H), 8.20 (s, 1H), 8.71 (d, 1H), 9.33 (d, 1H).

ESI/MS (m/e, %): 278 [(M+1)⁺, 100].

### Example 21

### 5-(2-Furyl)-2-methyl-6-pyridin-4-yl-3H-imidazo[4,5-b]pyridine

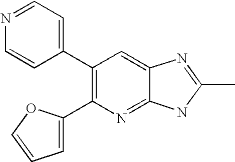

A mixture of 2-(2-furyl)-3,4′-bipyridine-5,6-diamine (Example 14) (0.100 g, 0.396 mmol) and 1,1,1-triethoxyethane (0.144 mL, 0.800 mmol) in 4 mL of acetic acid was heated at 140° C. for 2 hours in a sealed tube. Water was added and the pH was adjusted to 6-7 with 5% aqueous sodium hydrogen carbonate. The mixture was extracted with ethyl acetate and the organic layer was dried and evaporated. The crude mixture was purified by flash chromatography (95:5 dichloromethane/methanol) to give the title compound (0.055 g, 47%).

δ ¹H NMR (CDCl₃): 2.56 (s, 3H), 6:10-6.18 (dd, 1H), 6.37-6.39 (m, 1H), 7.27-7.31 (m, 2H), 7.42.7.43 (m, 1H), 7.86 (s, 1H), 8.66-8.68 (m, 2H).

ESI/MS (m/e, %): 277 [(M+1)⁺, 100].

### Example 22

### 2-cyclopropyl-5-(2-furyl)-6-pyrimidin-4-yl-3H-imidazo[4,5-b]pyridine

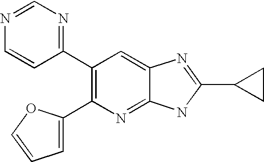

To a stirred solution of 6-(2-furyl)-5-pyrimidin-4-ylpyridine-2,3-diamine (Example 16) (50 mg, 0.197 mmol) in THF (3 mL), was added triethylamine (30 μL, 0.217 mmol) and the mixture was cooled to −10° C. Cyclopropanecarbonyl chloride (21 mg, 0.197 mmol) was added dropwise and the mixture was stirred for 30 minutes. The mixture was evaporated and glacial acetic acid (1 mL) was added and heated in a sealed tube to 140° C. After stirring for one day, the mixture was cooled and taken up in a small amount of water and taken to pH 7 with saturated aqueous sodium hydrogen carbonate solution and then extracted with ethyl acetate. The organic layer was dried and evaporated and the residue purified by flash chromatography (98:2 dichloromethane/methanol) to give the title compound as a solid (11 mg, 18%).

ESI/MS (m/e, %): 304 [(M+1)⁺, 100].

### Example 23

### 2-Cyclopropyl-5-(2-furyl)-6-pyridin-4-yl-3H-imidazo[4,5-b]pyridine

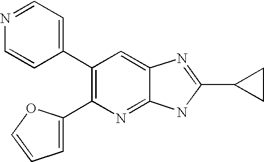

To a solution of 2-(2-furyl)-3,4′-bipyridine-5,6-diamine (Example 14) (0.078 g, 0.31 mmol) and triethylamine (0.045 mL, 0.34 mmol) in 4 mL of tetrahydrofuran at −10° C., was added cyclopropanecarbonyl chloride (0.028 mL, 0.31 mmol). The mixture was stirred at this temperature for 30 minutes and the solvent was evaporated. The crude mixture was partitioned between dichloromethane and 5% aqueous sodium hydrogen carbonate. The organic layer was dried and evaporated to give N-[6-amino-2-(2-furyl)-3,4′-bipyridin-5-yl]cyclopropanecarboxamide (0.064 g) which was used in the next step without further purification.

A solution of N-[6-amino-2-(2-furyl)-3,4′-bipyridin-5-yl]cyclopropanecarboxamide (0.052 g, 0.163 mmol) in 3 mL of acetic acid was heated at 140° C. for 14 hours in a sealed tube. The mixture was concentrated and purified by flash chromatography (98:2 dichloromethane/methanol) to give the title compound (0.005 g, 19%).

ESI/MS (m/e, %): 303 [(M+1)⁺, 100].

### Example 24

### 5-(2-Furyl)-6-pyridin-4-yl-3H-imidazo[4,5-b]pyridine

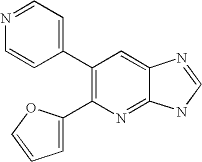

A mixture of intermediate 2-(2-furyl)-3,4′-bipyridine-5,6-diamine (Example 14) (0.050 g, 0.200 mmol) and diethoxymethoxyethane (0.066 mL, 0.400 mmol) in acetic acid (2 mL) was heated at 140° C. for 1 hour in a sealed tube. Water was added and the pH was adjusted to 6-7 with 5% aqueous sodium hydrogen carbonate. The mixture was extracted with ethyl acetate and the organic layer was dried and evaporated. The crude mixture was purified by flash chromatography (100:8 dichloromethane/methanol) to give the title compound (0.032 g, 61%).

δ ¹H NMR (DMSO): 5.75 (s, 1H), 6.43-6.51 (m, 2H), 7.29-7.32 (m, 2H), 7.55 (s, H), 7.99 (s, 1H), 8.56-8.59 (m, 2H).

ESI/MS (m/e, %): 263 [(M+1)⁺, 100].

### Example 25

### 5-(2-Furyl)-6-[2-(methylthio)pyrimidin-4-yl]-3H-imidazo[4,5-b]pyridine

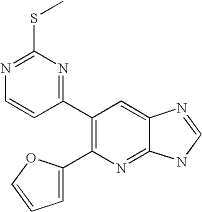

A mixture of 6-(2-furyl)-5-[2-(methylthio)pyrimidin-4-yl]pyridine-2,3-diamine (Example 15) (40 mg, 0.13 mmol) and triethylorthoformate (542 mg, 3.66 mmol) was heated in a sealed tube to 140° C. After stirring for 1 hour, the mixture was cooled and taken up in a small amount of water and then extracted with ethyl acetate. The organic layer was dried and evaporated. The residue was purified by flash chromatography (98:2 dichloromethane/methanol) to give the title compound as a solid (10 mg, 25%).

ESI/MS (m/e, %): 310 [(M+1)⁺, 100].

### Example 26

### 5-(2-Furyl)-1-methyl-6-pyrimidin-4-yl-1,3-dihydro-2H-imidazo[4,5-b]pyridin-2-one

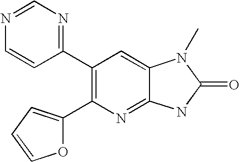

A mixture of 3-(2,4-Dimethoxybenzyl)-5-furan-2-yl-1-methyl-6-pyrimidin-4-yl-1,3-dihydro-imidazo[4,5-b]pyridin-2-one (Intermediate 16) (0.100 g, 0.23 mmol), trifluoroacetic acid (5 mL) and thioanisole (1.3 mL) was heated to 65° C. and left overnight. The mixture was concentrated, neutralised with 4% aqueous sodium hydrogen carbonate solution and extracted with ethyl acetate. The organic layer was dried (MgSO₄), evaporated and the residue purified by flash chromatography (dichloromethane to 25:1 dichloromethane/methanol) to give the title compound (0.021 g, 32%) as a pale yellow solid.

δ ¹H NMR (CDCl₃+CD₃OD): 9.28 (d, 1H), 8.60 (d, 1H), 7.50 (s, 1H), 7.30 (d, 1H), 7.10 (dd, 1H), 6.63 (dd, 1H), 6.42 (dd, 1H), 3.40 (s, 3H).

ESI/MS (m/e, %): 294 [(M+1)⁺, 100].

### Example 27

### 6-(2-furyl)-5-pyrimidin-4-yl-1H-pyrazolo[3,4-b]pyridine

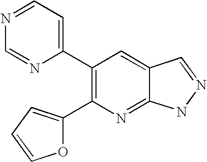

A solution of 6-(2-furyl)-5-pyrimidin-4-yl-1H-pyrazolo[3,4-b]pyridin-3-amine (2.0 g, 7.2 mmol) (Example 48) in a mixture of glacial acetic acid (10 mL), water (4.3 mL) and concentrated aqueous hydrochloric acid (1.2 mL) was cooled to 0° C. and a solution of sodium nitrite (0.595 g, 8.6 mmol) in water (2 mL) was added dropwise. The mixture was stirred for 30 minutes and then a 50% aqueous solution of hypophosphorous acid (11.3 mL) was added dropwise and the mixture was stirred a further 6 hours at 0° C. The mixture was neutralized with 6M aqueous sodium hydroxide solution and the solid that formed was filtered and purified by flash chromatography (2:1 hexanes/ethyl acetate to ethyl acetate) to give the title compound (0.78 g, 41%) as an off-white solid.

δ ¹H NMR (DMSO): 13.81 (s, 1H), 9.31 (d, 1H), 8.85 (d, 1H), 8.51 (s, 1H), 8.33 (s, 1H), 7.63 (dd, 1H), 7.46 (dd, 1H), 6.87 (dd, 1H), 6.63 (dd, 1H).

ESI/MS (m/e, %): 264 [(M+1)⁺, 100].

### Example 28

### 3-Chloro-6-(2-furyl)-5-pyrimidin-4-yl-1H-pyrazolo[3,4-b]pyridine

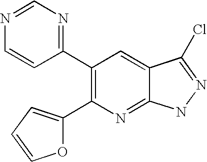

A suspension 6-(2-furyl)-5-pyrimidin-4-yl-1,2-dihydro-3H-pyrazolo[3,4-b]pyridin-3-one (Example 31) (0.9 g, 3.22 mmol) in phosphorous oxychloride (5 mL) was heated in a sealed tube to 110° C. and stirred overnight. The mixture was then cooled and evaporated to dryness. Water was added and the pH was adjusted to 7 with saturated aqueous sodium hydrogen carbonate solution. The solid that formed was filtered and purified by flash chromatography (1:1 hexanes/ethyl acetate) to give the title compound (0.16 g, 17%) as a white solid.

δ ¹H NMR (CDCl₃): 11.89 (s, 1H), 9.36 (d, 1H), 8.76 (d, 1H), 8.28 (s, 1H), 7.53 (m, 1H), 7.32 (dd, 1H), 6.63 (dd, 1H), 6.50 (m, 1H)

ESI/MS (m/e, %): 298 [(M+1)⁺, 100].

### Example 29

### 3-ethoxy-6-(2-furyl)-5-pyrimidin-4-yl-1H-pyrazolo[3,4-b]pyridine

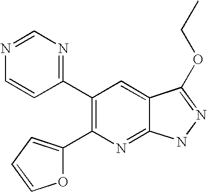

A mixture of 3-Ethoxy-6-furan-2-yl-1-(4-methoxybenzyl)-5-pyrimidin-4-yl-1H-pyrazolo[3,4-b]pyridine (Intermediate 21) (0.053 g, 0.12 mmol), trifluoroacetic acid (2.5 mL) and thioanisole (0.7 mL) was heated to 80° C. and left overnight. The mixture was concentrated, neutralised with 4% aqueous sodium hydrogen carbonate solution and extracted with ethyl acetate. The organic layer was dried (MgSO₄), evaporated and the residue purified by flash chromatography (hexanes to 1:1 hexanes/ethyl acetate) to give the title compound (0.010 g, 27%) as a light brown solid.

δ ¹H NMR (CDCl₃): 10.74 (s, 1H), 9.31 (d, 1H), 8.70 (d, 1H), 8.30 (s, 1H), 7.46 (m, 1H), 7.25 (dd, 1H), 6.66 (dd, 1H), 6.48 (m, 1H).

ESI/MS (m/e, %): 308 [(M+1)⁺, 100].

### Example 30

### 6-(2-furyl)-5-[2-(methylthio)pyrimidin-4-yl]-1H-pyrazolo[3,4-b]pyridin-3-amine

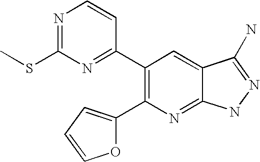

To a suspension 2-chloro-6-(2-furyl)-5-[2-(methylthio)pyrimidin-4-yl]nicotinonitrile (Intermediate 12) (1.00 g, 3.0 mmol) in ethyl alcohol (20 mL) was added hydrazine monohydrate (0.31 g, 9.0 mmol) and the mixture was heated to reflux and stirred overnight. The mixture was treated with water and the precipitate that formed was filtered, washed with water and diethyl ether and dried in vacuo to give the title compound (0.90 g, 91%) as a yellow solid.

δ ¹H NMR (DMSO): 12.28 (s, 1H), 8.57 (d, 1H), 8.44 (s, 1H), 7.62 (dd, 1H), 6.98 (d, 1H), 6.81 (dd, 1H), 6.60 (dd, 1H), 5.84 (s, 2H), 2.43 (s, 3H).

ESI/MS (m/e, %): 325 [(M+1)⁺, 100].

### Example 31

### 6-(2-furyl)-5-pyrimidin-4-yl-1,2-dihydro-3H-pyrazolo[3,4-b]pyridin-3-one

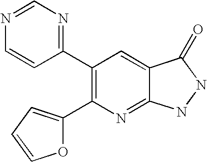

To a stirred suspension of methyl 2-chloro-6-(2-furyl)-5-pyrimidin-4-ylnicotinate (Intermediate 14) (3.0 g, 9.52 mmol) in ethanol (100 mL) was added hydrazine monohydrate (4.77 g, 95.2 mmol) and the mixture was heated to 95° C. in a sealed tube. After stirring overnight, the mixture was filtered hot to remove a small amount of an insoluble dark solid and the cooled filtrate was evaporated. The residue was triturated with ethanol and the solid was filtered and dried to give the title compound (2.44 g, 92%) as an off-white solid.

δ ¹H NMR (DMSO): 9.17 (d, 1H), 8.74 (d, 1H), 8.27 (s, 1H), 7.58 (m, 1H), 7.35 (dd, 1H), 6.76 (dd, 1H), 6.57 (m, 1H).

ESI/MS (m/e, %): 280 [(M+1)⁺, 100].

### Example 32

### 6-(2-furyl)-5-[2-(methylthio)pyrimidin-4-yl]-1H-pyrazolo[3,4-b]pyridine

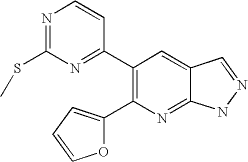

A solution of 6-(2-furyl)-5-[2-(methylthio)pyrimidin-4-yl]-1H-pyrazolo[3,4-b]pyridin-3-amine (Example 30) (0.60 g, 1.85 mmol) in a mixture of glacial acetic acid (3 mL), water (1.3 mL) and concentrated aqueous hydrochloric acid (0.34 mL) was cooled to 0° C. and a solution of sodium nitrite (0.153 g, 2.22 mmol) in water (0.5 mL) was added dropwise. The mixture was stirred for 30 minutes and then a 50% aqueous solution of hypophosphorous acid (3.4 mL) was added dropwise and the mixture was stirred a further 6 hours at 0° C. The mixture was neutralised with 6M aqueous sodium hydroxide solution and the solid that formed was filtered and purified by flash chromatography (3:1 hexanes/ethyl acetate to 2:1 hexanes/ethyl acetate) to give the title compound (0.24 g, 42%) as a white solid.

δ ¹H NMR (DMSO): 8.61 (d, 1H), 8.47 (s, 1H), 8.24 (s, 1H), 7.62 (dd, 1H), 7.14 (d, 1H), 6.82 (dd, 1H), 6.60 (dd, 1H), 2.21 (s, 3H).

ESI/MS (m/e, %): 310 [(M+1)⁺, 100].

### Example 33

### 6-(2-Furyl)-5-(2-methoxypyrimidin-4-yl)-1H-pyrazolo[3,4-b]pyridine

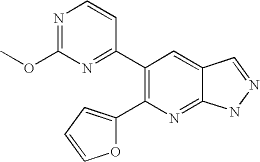

To a suspension of 6-(2-furyl)-5-[2-(methylsulfonyl)pyrimidin-4-yl]-1H-pyrazolo[3,4-b]pyridine (Intermediate 13) (0.070 g, 0.21 mmol) in methanol (2 mL) under an atmosphere of argon was added, in portions, 60% sodium hydride as a suspension in mineral oil (0.025 g, 0.62 mmol). The reaction vial was capped and warmed to 70° C. After 3 hours, the reaction was cooled and diluted with water and the pH was adjusted to 5-6 using 2M aqueous hydrochloric acid. The precipitate was filtered, washed with water and hexanes and dried to give the title compound (0.028 g, 46%) as a white solid.

δ ¹H NMR (DMSO): 8.61 (d, 1H), 8.46 (s, 1H), 8.23 (s, 1H), 7.61 (dd, 1H), 7.11 (d, 1H), 6.82 (dd, 1H), 6.60 (dd, 1H), 3.79 (s, 3H).

ESI/MS (m/e, %): 294 [(M+1)⁺, 100].

### Example 34

### N-cyclopropyl-4-[6-(2-furyl)-1H-pyrazolo[3,4-b]pyridin-5-yl]pyrimidin-2-amine

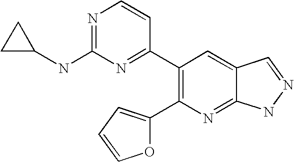

To a suspension of 6-(2-furyl)-5-[2-(methylsulfonyl)pyrimidin-4-yl]-1H-pyrazolo[3,4-b]pyridine (Intermediate 13) (0.050 g, 0.15 mmol) in acetonitrile (1 mL) and triethylamine (0.051 g, 0.50 mmol) was added cyclopropylamine (0.059 g, 1.18 mmol). The reaction vial was capped and warmed to 100° C. After stirring overnight, the reaction was cooled and filtered. The filtrate was evaporated and the residue purified by flash chromatography (1:1 hexanes/ethyl acetate) to give the title compound (0.012 g, 26%) as a white solid.

δ ¹H NMR (DMSO): 8.32 (s, 1H), 8.30 (d, 1H), 8.21 (s, 1H), 7.67 (dd, 1H), 7.43 (d, 1H), 6.66 (dd, 1H), 6.55 (dd, 1H), 1.27 (m, 1H), 0.58 (m, 2H), 0.42 (m, 2H).

ESI/MS (m/e, %): 319 [(M+1)⁺, 100].

### Example 35

### 4-[6-(2-furyl)-1H-pyrazolo[3,4-b]pyridin-5-yl]-N-isopropylpyrimidin-2-amine

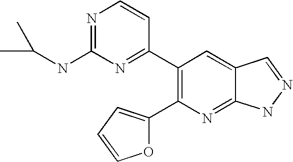

To a suspension of 6-(2-furyl)-5-[2-(methylsulfonyl)pyrimidin-4-yl]-1H-pyrazolo[3,4-b]pyridine (Intermediate 13) (0.050 g, 0.15 mmol) in acetonitrile (1 mL) and triethylamine (0.016 g, 0.16 mmol) was added isopropylamine (0.174 g, 2.94 mmol). The reaction vial was capped and warmed to 100° C. After stirring overnight, the reaction was cooled and evaporated. The residue was purified by flash chromatography (95:5 dichloromethane/methanol) to give the title compound (0.007 g, 15%) as a white solid.

δ ¹H NMR (CD₃OD): 8.38 (s, 1H), 8.24 (d, 1H), 8.17 (s, 1H), 7.50 (dd, 1H), 6.81 (dd, 1H), 6.53 (m, 2H), 4.07 (m, 1H), 1.23 (s, 3H), 1.18 (s, 3H).

ESI/MS (m/e, %): 321 [(M+1)⁺, 100].

### Example 36

### 5-(2-ethoxypyrimidin-4-yl)-6-(2-furyl)-1H-pyrazolo[3,4-b]pyridine

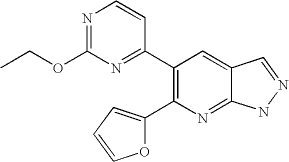

To a suspension of 6-(2-furyl)-5-[2-(methylsulfonyl)pyrimidin-4-yl]-1H-pyrazolo[3,4-b]pyridine (Intermediate 13) (0.070 g, 0.21 mmol) in ethanol (2 mL) under an atmosphere of argon was added, in portions, 60% sodium hydride as a suspension in mineral oil (0.025 g, 0.62 mmol). The reaction vial was capped and warmed to 70° C. After 3 hours, the reaction was cooled and diluted with water and the pH was adjusted to 5-6 using 2M aqueous hydrochloric acid. The precipitate was filtered, washed with water and hexanes and dried to give the title compound (0.028 g, 44%) as a white solid.

δ ¹H NMR (CD₃OD): 8.54 (d, 1H), 8.40 (s, 1H), 8.19 (s, 1H), 7.43 (dd, 1H), 7.01 (d, 1H), 6.92 (dd, 1H), 6.57 (dd, 1H), 4.36 (q, 2H), 1.32 (t, 3H).

ESI/MS (m/e, %): 308 [(M+1)⁺, 100].

### Example 37

### 6-(2-Furyl)-5-(2-isopropoxypyrimidin-4-yl)-1H-pyrazolo[3,4-b]pyridine

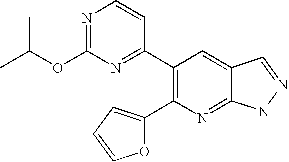

To a suspension of 6-(2-furyl)-5-[2-(methylsulfonyl)pyrimidin-4-yl]-1H-pyrazolo[3,4-b]pyridine (Intermediate 13) (0.087 g, 0.26 mmol) in isopropanol (2 mL) under an atmosphere of argon was added, in portions, 60%-sodium hydride as a suspension in mineral oil (0.031 g, 0.77 mmol). The reaction vial was capped and warmed to 70° C. After 3 hours, the reaction was cooled and diluted with water and the pH was adjusted to 5-6 using 2M aqueous hydrochloric acid. The precipitate was filtered, washed with water and hexanes and dried to give the title compound (0.040 g, 49%) as a white solid.

δ ¹H NMR (CD₃OD): 8.52 (d, 1H), 8.39 (s, 1H), 8.20 (s, 1H), 7.44 (dd, 1H), 7.05 (d, 1H), 6.89 (dd, 1H), 6.56 (dd, 1H), 5.22 (m, 1H), 1.32 (s, 3H), 1.29 (s, 3H).

ESI/MS (m/e, %): 322 [(M+1)⁺, 100].

### Example 38

### 5-[2-(Cyclohexyloxy)pyrimidin-4-yl]-6-(2-furyl)-1H-pyrazolo[3,4-b]pyridine

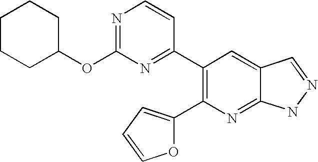

To a suspension of 6-(2-furyl)-5-[2-(methylsulfonyl)pyrimidin-4-yl]-1H-pyrazolo[3,4-b]pyridine (Intermediate 13) (0.080 g, 0.23 mmol) in tetrahydrofuran (1 mL) under an atmosphere of argon was added cyclohexanol (0.071 g, 0.70 mmol) followed by, in portions, 60% sodium hydride as a suspension in mineral oil (0.028 g, 0.70 mmol). The reaction vial was capped and warmed to 70° C. After 4 hours, the reaction was cooled and diluted with water and the pH was adjusted to 5-6 using 2M aqueous hydrochloric acid. The precipitate was filtered, washed with water and hexanes and dried to give the title compound (0.044 g, 52%) as a white solid.

δ ¹H NMR (CD₃OD): 8.53 (d, 1H), 8.39 (s, 1H), 8.20 (s, 1H), 7.42 (dd, 1H), 7.05 (d, 1H), 6.89 (dd, 1H), 6.51 (dd, 1H), 2.01-1.32 (m, 11H).

ESI/MS (m/e, %): 362 [(M+1)⁺, 100].

### Example 39

### 6-(2-Furyl)-N-isobutyl-5-pyrimidin-4-yl-1H-pyrazolo[3,4-b]pyridin-3-amine

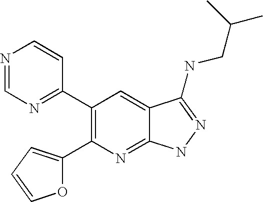

To a suspension of 6-(2-furyl)-5-pyrimidin-4-yl-1H-pyrazolo[3,4-b]pyridin-3-amine (Example 48) (0.060 g, 0.22 mmol) in dichloroethane (2.5 mL) and glacial acetic acid (0.074 mL) was added 2-methyl-propionaldehyde (0.22 mmol) and sodium triacetoxyborohydride (0.128 g, 0.60 mmol). After stirring for 4 days at room temperature, the mixture was partitioned between ethyl acetate and 4% aqueous sodium hydrogen carbonate solution. The organic layer was washed with brine, dried (MgSO₄) and evaporated and the residue was purified by flash chromatography (1:1 ethyl acetate/hexanes to 2:1 ethyl acetate/hexanes) to give the title compound (0.030 g, 42%) as an off-white solid.

δ ¹H NMR (DMSO): 12.23 (s, 1H), 9.18 (d, 1H), 8.76 (d, 1H), 8.51 (s, 1H), 7.58 (dd, 1H), 7.23 (dd, 1H), 6.81 (dd, 1H), 6.52 (dd, 1H), 6.47 (s, 1H), 3.10 (t, 2H), 1.93 (m, 1H), 1.02 (d, 6H).

ESI/MS (m/e, %): 335 [(M+1)⁺, 100].

### Example 40

### N-{6-(2-furyl)-5-[2-(methylthio)pyrimidin-4-yl]-1H-pyrazolo[3,4-b]pyridin-3-yl}acetamide

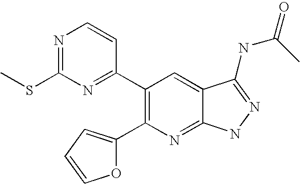

To a suspension of 6-(2-furyl)-5-[2-(methylthio)pyrimidin-4-yl]-1H-pyrazolo[3,4-b]pyridin-3-amine (Example 30) (0.10 g, 0.31 mmol) in pyridine (0.5 mL) was added acetic anhydride (0.032 mL, 0.34 mmol) and the mixture was heated to reflux. After 20 hours the mixture was cooled and poured into water. The precipitate was filtered, washed with water and dried in the air to give the title compound (0.081 g, 72%) as an off-white solid

δ ¹H NMR (DMSO): 13.51 (s, 1H), 10.82 (NH), 8.63 (d, 1H), 8.61 (s, 1H), 7.63 (dd, 1H), 7.10 (d, 1H), 6.82 (dd, 1H), 6.58 (dd, 1H), 2.38 (s, 3H), 2.08 (s, 3H).

ESI/MS (m/e, %): 367 [(M+1)⁺, 100].

### Example 41

### 6-(3-Fluorophenyl)-5-pyrimidin-4-yl-1H-pyrazolo[3,4-b]pyridin-3-amine

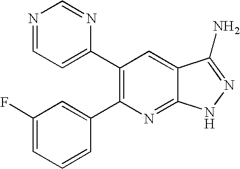

A solution of 2-chloro-6-(3-fluorophenyl)-5-pyrimidin-4-ylnicotinonitrile (Intermediate 15) (16.86 g, 53.9 mmol) and hydrazine monohydrate (9.15 mL, 190 mmol) in ethanol (200 mL) was heated to 80° C. After 15 hours the mixture was cooled and the precipitate was filtered and washed with water. The filtrate was evaporated, water was added and the precipitate was filtered. The solid was dried to give the title compound (13.75 g, 97%) as a yellow solid.

δ ¹H NMR (DMSO): 5.89 (s, 2H), 7.04 (d, 1H), 7.15 (dd, 1H), 7.19 (m, 2H), 7.34 (dd, 1H), 8.60 (d, 2H), 9.17 (s, 1H), 12.33 (s, 1H).

### Example 42

### 6-(3-Fluorophenyl)-5-pyrimidin-4-yl-1H-pyrazolo[3,4-b]pyridine

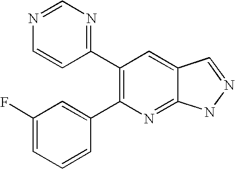

A solution of 6-(3-fluorophenyl)-5-pyrimidin-4-yl-1H-pyrazolo[3,4-b]pyridin-3-amine (Example 41) (0.146 g, 0.47 mmol) in a mixture of glacial acetic acid (0.75 mL), water (0.33 mL) and concentrated aqueous hydrochloric acid (0.085 mL) was cooled to 0° C. and a solution of sodium nitrite (0.039 g, 0.57 mmol) in water (0.2 mL) was added dropwise. The mixture was stirred for 30 minutes and then a 50% aqueous solution of hypophosphorous acid (0.86 mL) was added dropwise and the mixture was stirred for a further 60 minutes at 0° C. The mixture was neutralised with 6M aqueous sodium hydroxide solution and the solid that formed was filtered and purified by flash chromatography (95:5 dichloromethane/methanol) to give the title compound (0.070 g, 51%) as an off-white solid.

δ ¹H NMR (DMSO): 9.16 (d, 1H), 8.70 (d, 1H), 8.60 (s, 1H), 8.33 (s, 1H), 7.38-7.18 (m, 4H), 7.06 (m, 1H).

ESI/MS (m/e, %): 292 [(M+1)⁺, 100].

### Example 43

### 6-(2-Furyl)-5-pyrimidin-4-yl-1H-pyrrolo[2,3-b]pyridine

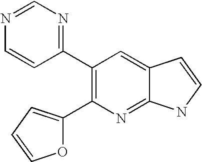

To a solution of 3-ethynyl-6-(2-furyl)-5-pyrimidin-4-ylpyridin-2-amine (Intermediate 18) (0.50 g, 1.9 mmol) in 1-methyl-2-pyrrolidinone (12 mL) was added potassium tert-butoxide (0.45 g, 4.0 mmol) and the mixture was heated to 70° C. in a sealed tube. After 48 hours, the mixture was cooled, diluted with water and extracted with ethyl acetate. The organic layer was washed with brine, dried (MgSO₄) and evaporated. The residue was purified by ion-exchange chromatography using an SCX column (eluting with methanol then 7M ammonia in ethanol) to give the title compound (0.39 g, 78%) as a beige solid.

δ ¹H NMR (CDCl₃): 11.28 (s, 1H), 9.33 (d, 1H), 8.67 (d, 1H), 8.25 (s, 1H), 7.46 (m, 1H), 7.40 (dd, 1H), 7.23 (m, 1H), 6.61 (dd, 1H), 6.50 (d, 2H)

ESI/MS (m/e, %): 263 [(M+1)⁺, 100].

### Example 44

### 2-(3-Fluorophenyl)-6-(2-furyl)-5-pyrimidin-4-yl-1H-pyrrolo[2,3-b]pyridine

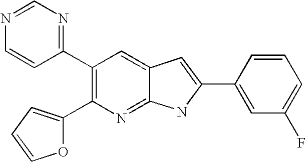

To a solution of 3-[(3-fluorophenyl)ethynyl]-6-(2-furyl)-5-pyrimidin-4-ylpyridin-2-amine (Intermediate 20) (0.145 g, 0.41 mmol) in 1-methyl-2-pyrrolidinone (7 mL) was added potassium tert-butoxide (0.096 g, 0.85 mmol) and the mixture was heated to 70° C. in a sealed tube. After 72 hours, the mixture was cooled, diluted with water and extracted with ethyl acetate. The organic layer was washed with brine, dried (MgSO₄) and evaporated. The residue was purified by flash chromatography (1:1 hexanes/ethyl acetate) to give the title compound (0.095 g, 66%) as a yellow solid.

δ ¹H NMR (CDCl₃): 10.06 (s, 1H), 9.33 (d, 1H), 8.68 (d, 1H), 8.20 (s, 1H), 7.40-7.20 (m, 5H), 7.03 (m, 1H), 6.86 (dd, 1H), 6.50 (dd, 1H), 6.33 (m, 1H).

ESI/MS (m/e, %): 357 [(M+1)⁺, 100].

### Example 45

### 6-(2-Furyl)-2-phenyl-5-pyrimidin-4-yl-1H-pyrrolo[2,3-b]pyridine

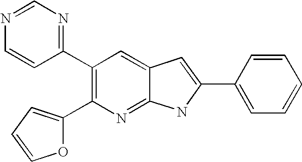

To a solution of 6-(2-furyl)-3-(phenylethynyl)-5-pyrimidin-4-ylpyridin-2-amine (Intermediate 19) (0.04 g, 0.12 mmol) in 1-methyl-2-pyrrolidinone (2 mL) was added potassium tert-butoxide (0.030 g, 0.25 mmol) and the mixture was heated to 70° C. in a sealed tube. After 72 hours, the mixture was cooled, diluted with water and extracted with ethyl acetate. The organic layer was washed with brine, dried (MgSO₄) and evaporated. The residue was purified by flash chromatography (1:1 hexanes/ethyl acetate) to give the title compound (0.030 g, 75%) as a pale yellow solid.

δ ¹H NMR (CDCl₃): 9.45 (s, 1H), 9.33 (d, 1H), 8.66 (d, 1H), 8.17 (s, 1H), 7.68 (m, 2H), 7.51-7.21 (m, 5H), 6.86 (dd, 1H), 6.51 (dd, 1H), 6.40 (m, 1H)

ESI/MS (m/e, %): 339 [(M+1)⁺, 100].

### Example 46

### 6-(5-Bromo-2-furyl)-3-chloro-5-pyrimidin-4-yl-1H-pyrazolo[3,4-b]pyridine

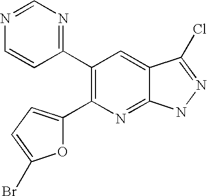

To a solution of 3-chloro-6-(2-furyl)-5-pyrimidin-4-yl-1H-pyrazolo[3,4-b]pyridine (Example 28) (0.12 g, 0.40 mmol) in chloroform (1 mL) and acetonitrile (0.4 mL) was added bromine (0.44 mmol). The mixture was stirred for 48 hours and then the mixture was diluted with chloroform and the organic layer was washed with 4% aqueous sodium hydrogen carbonate solution and 5% aqueous sodium thiosulphate solution. The organic layer was dried (MgSO₄), evaporated and the residue purified by flash chromatography (98:2 dichloromethane/methanol) to give the title compound (13.4 mg, 9%) as an off-white solid.

δ 5 ¹H NMR (DMSO): 9.23 (d, 1H), 8.90 (d, 1H), 8.36 (s, 1H), 7.69 (dd, 1H), 6.79 (d, 1H), 6.71 (d, 1H).

ESI/MS (m/e, %): 378 [(M+1)⁺, 100].

### Example 47

### 5-(5-Bromo-2-furyl)-6-pyrimidin-4-yl-1,3-dihydro-2H-imidazo[4,5-b]pyridin-2-one

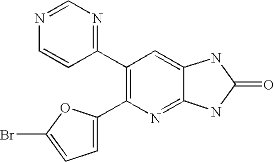

To a solution of 5-(2-furyl)-6-pyrimidin-4-yl-1,3-dihydro-2H-imidazo[4,5-b]pyridin-2-one (Example 12) (0.36 g, 1.32 mmol) in 7 mL of acetic acid was added bromine (0.075 mL, 1.45 mmol). The mixture was heated at 60° C. for 2 hours then cooled to room temperature. The mixture was partially concentrated and the precipitate that formed was filtered off to give the title compound (0.440 g, 92%).

ESI/MS (m/e, %): 359 [(M+1)⁺, 100].

### Example 48

### 6-(2-Furyl)-5-pyrimidin-4-yl-1H-pyrazolo[3,4-b]pyridin-3-amine

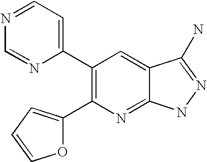

To a suspension of 2-chloro-6-(2-furyl)-5-pyrimidin-4-ylnicotinonitrile (Intermediate 22) (1.14 g, 4.0 mmol) in ethyl alcohol (20 mL) was added hydrazine monohydrate (0.61 g, 12.1 mmol) and the mixture was heated to reflux and stirred overnight. The mixture was treated with 4% aqueous sodium hydrogen carbonate solution and the precipitate was filtered, washed with water, ethyl acetate and ethanol and dried in vacuo to give the title compound (0.80 g, 71%) as an orange solid.

δ ¹H NMR (DMSO): 5.83 (s, 2H), 6.58 (dd, 1H), 6.80 (dd, 1H), 7.25 (dd, 1H), 7.60 (dd, 1H), 8.42 (s, 1H), 8.74 (d, 1H), 9.20 (d, 1H), 12.25 (s, 1H).

ESI/MS (m/e, %): 279 [(M+1)⁺, 100].

### Example 49

### N-[6-(2-Furyl)-5-pyrimidin-4-yl-1H-pyrazolo[3,4-b]pyridin-3-yl]acetamide

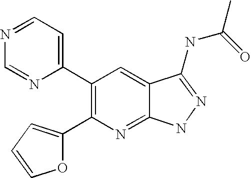

To a suspension of 6-(2-furyl)-5-pyrimidin-4-yl-1H-pyrazolo[3,4-b]pyridin-3-amine (Example 48) (0.101 g, 0.36 mmol) in pyridine (0.5 mL) was added acetic anhydride (0.038 mL, 0.4 mmol) and the mixture was heated to reflux. After 20 hours the mixture was cooled and poured into water. The precipitate was filtered, washed with water and dried in the air to give the title compound (0.084 g, 72%) as an orange solid.

δ ¹H NMR (DMSO): 2.10 (s, 3H), 6.59 (dd, 1H), 6.79 (dd, 1H), 7.38 (dd, 1H), 7.61 (dd, 1H), 8.60 (s, 1H), 8.78 (d, 1H), 9.22 (d, 1H), 10.86 (s, 1H), 13.46 (s, 1H).

ESI/MS (m/e, %): 321 [(M+1)⁺, 100].

### EXAMPLES

TABLE 2

| Example | Structure |
|---|---|
| 1 | 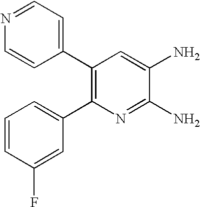 |
| 2 | 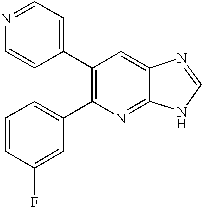 |
| 3 | 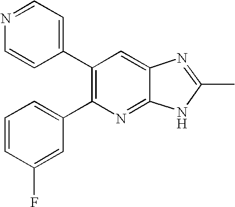 |
| 4 | 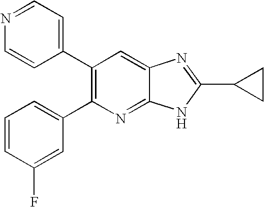 |
| 5 | 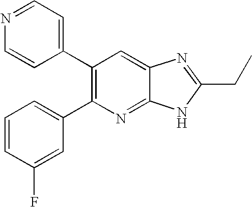 |
| 6 | 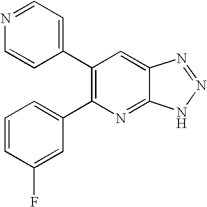 |
| 7 | 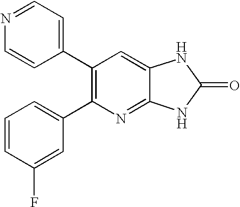 |
| 8 | 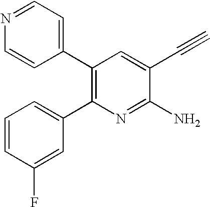 |
| 9 | 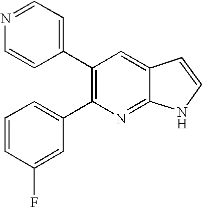 |
| 10 | 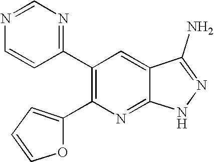 |
| 11 | 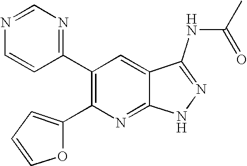 |
| 12 | 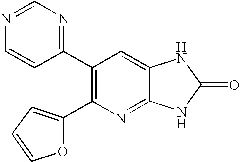 |
| 13 | 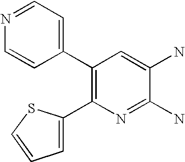 |
| 14 | 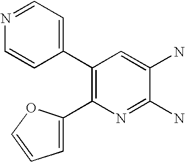 |
| 15 | 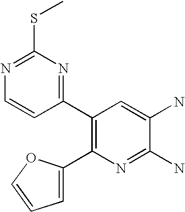 |
| 16 | 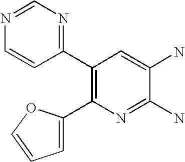 |
| 17 | 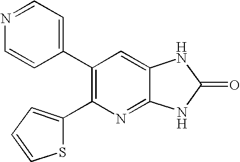 |
| 18 | 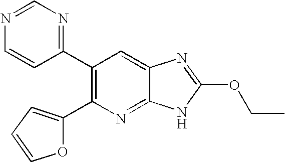 |
| 19 | 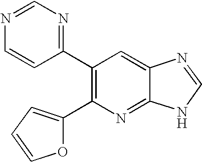 |
| 20 | 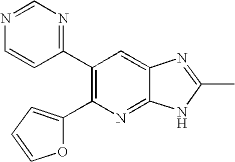 |
| 21 | 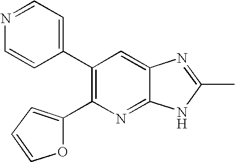 |
| 22 | 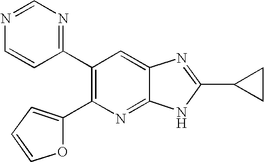 |
| 23 | 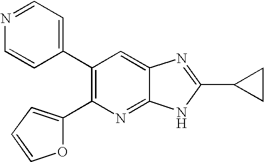 |
| 24 | 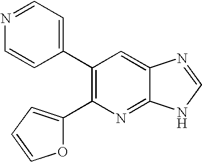 |
| 25 | 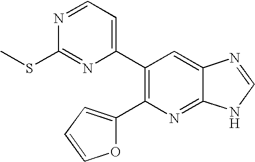 |
| 26 | 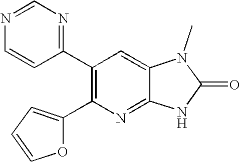 |
| 27 | 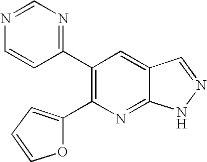 |
| 28 | 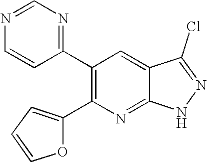 |
| 29 | 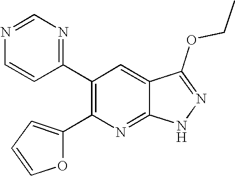 |
| 30 | 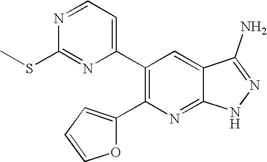 |
| 31 | 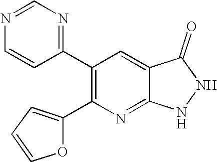 |
| 32 | 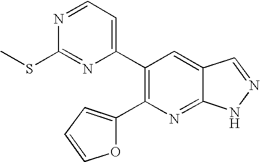 |
| 33 | 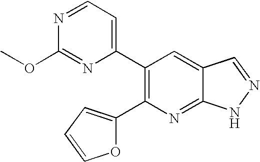 |
| 34 | 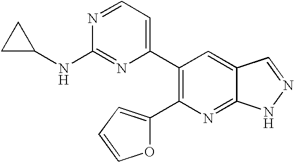 |
| 35 | 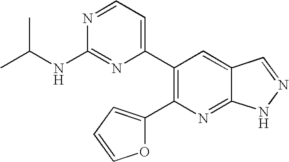 |
| 36 | 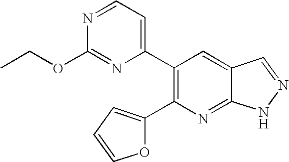 |
| 37 | 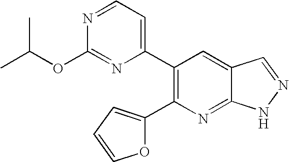 |
| 38 | 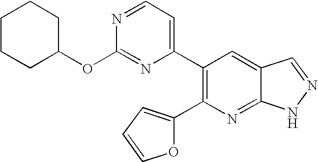 |
| 39 | 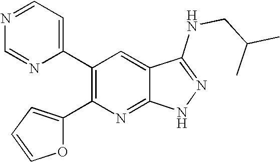 |
| 40 | 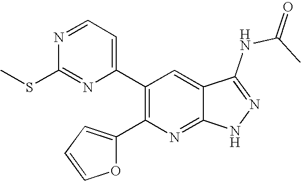 |
| 41 | 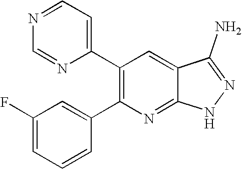 |
| 42 | 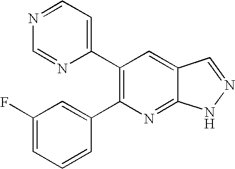 |
| 43 | 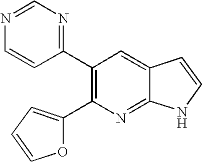 |
| 44 | 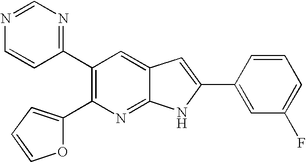 |
| 45 | 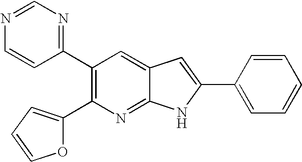 |
| 46 | 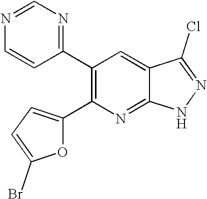 |
| 47 | 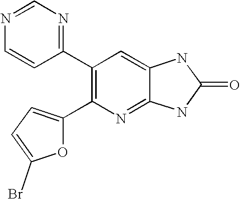 |
| 48 | 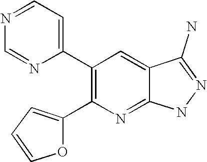 |
| 49 | 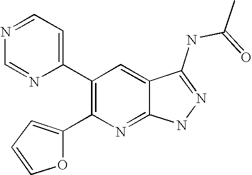 |

### Composition Example 1

50,000 capsules, each containing 100 mg of 6-(2-furyl)-5-pyrimidin-4-yl-1H-pyrazolo[3,4b]pyridin-3-amine (active ingredient), were prepared according to the following formulation:

| | |
|---|---|
| Active ingredient | 5 Kg |
| Lactose monohydrate | 10 Kg |
| Colloidal silicon dioxide | 0.1 Kg |
| Corn starch | 1 Kg |
| Magnesium stearate | 0.2 Kg |

### Procedure

The above ingredients were sieved through a 60 mesh sieve, and were loaded into a suitable mixer and filled into 50,000 gelatine capsules.

### Composition Example 2

50,000 tablets, each containing 50 mg of 6-(2-furyl)-5-pyrimidin-4-yl-1H-pyrazolo[3,4b]pyridin-3-amine (active ingredient), were prepared from the following formulation:

| | |
|---|---|
| Active ingredient | 2.5 Kg |
| Microcrystalline cellulose | 1.95 Kg |
| Spray dried lactose | 9.95 Kg |
| Carboxymethyl starch | 0.4 Kg |
| Sodium stearyl fumarate | 0.1 Kg |
| Colloidal silicon dioxide | 0.1 Kg |

### Procedure

All the powders were passed through a screen with an aperture of 0.6 mm, then mixed in a suitable mixer for 20 minutes and compressed into 300 mg tablets using 9 mm disc and flat bevelled punches. The disintegration time of the tablets was about 3 minutes.

1. A method for treating a subject afflicted with a pathological condition or disease susceptible to amelioration by antagonism of the A_{2B} adenosine receptor comprising administering to said subject an effective amount of a compound of formula (I) 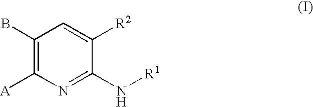
wherein:
A represents an optionally substituted monocyclic or polycyclic aryl or heteroaryl group,
B represents an optionally substituted monocyclic nitrogen-containing heterocyclic group,
and either
a) R¹ represents a hydrogen atom and R² represents a group chosen from —NH₂ and optionally substituted alkynyl groups
or
b) R², R¹ and the —NH— group to which R¹ is attached form a moiety chosen from the moieties of formulae (IIa), (IIb), (IIc), (IId) and (IIe): 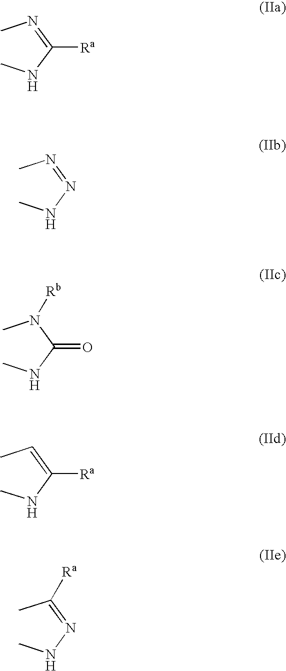
wherein:
R^{a} is a group chosen from a hydrogen atom, halogen atoms and groups chosen from optionally substituted alkyl, optionally substituted cycloalkyl, optionally substituted aryl, optionally substituted heteroaryl, —OR³, —SR³, —COOR³, —CONR³R⁴, —NR³R⁴, —NR³COR⁴ and —CN groups wherein R³ and R⁴ are independently chosen from a hydrogen atom and lower alkyl groups or cycloalkyl groups, and
R^{b} is a group chosen from a hydrogen atom and groups chosen from optionally substituted alkyl, optionally substituted cycloalkyl, optionally substituted aryl and optionally substituted heteroaryl groups.

2. A method according to claim 1, wherein B represents an optionally substituted monocyclic, six-membered heterocyclic ring having one or two nitrogen atoms.

3. A method according to claim 2, wherein B represents a group chosen from optionally substituted pyridines, optionally substituted pyrimidines, optionally substituted pyridazines and optionally substituted pyridinones.

4. A method according to claim 1, wherein the group B is unsubstituted or substituted with one group chosen from —OR³, —SR³, —R³ and —NHR³.

5. A method according to claim 1, wherein A represents an optionally substituted phenyl, furyl or thienyl group.

6. A method according to claim 1, wherein the group A is unsubstituted or substituted with one group chosen from halogen atoms and lower alkyl groups.

7. A method according to claim 1, wherein B represents a pyrimidinyl group and A represents a furyl group.

8. A method according to claim 1, wherein either R¹ represents a hydrogen atom, or R², R¹ and the —NH— group to which R¹ is attached, form a moiety chosen from the moieties of formulae (IIc) and (IIe).

9. A method according to claim 1, wherein R² represents an —NH₂ group or an optionally substituted alkynyl group.

10. A method according to claim 1, wherein R^{a} is chosen from lower alkyl groups and cycloalkyl groups.

11. A method according to claim 1, wherein R^{b} is chosen from lower alkyl groups and hydrogen atoms.

12. A method according to claim 1, wherein the compound is chosen from:
2-(3-Fluorophenyl)-3,4′-bipyridine-5,6-diamine,
5-(3-Fluorophenyl)-6-pyridin-4-yl-3H-imidazo[4,5-b]pyridine
5-(3-Fluorophenyl)-2-methyl-6-pyridin-4-yl-3H-imidazo[4,5-b]pyridine,
2-Cyclopropyl-5-(3-fluorophenyl)-6-pyridin-4-yl-3H-imidazo[4,5-b]pyridine,
2-Ethyl-5-(3-fluorophenyl)-6-pyridin-4-yl-3H-imidazo[4,5-b]pyridine,
5-(3-Fluorophenyl)-6-pyridin-4-yl-3H-[1,2,3]triazolo[4,5-b]pyridine
5-(3-Fluorophenyl)-6-pyridin-4-yl-1,3-dihydro-2H-imidazo[4,5-b]pyridin-2-one,
5-Ethynyl-2-(3-fluorophenyl)-3,4′-bipyridin-6-amine,
6-(3-Fluorophenyl)-5-pyridin-4-yl-1H-pyrrolo[2,3-b]pyridine,
6-(2-Furyl)-5-pyrimidin-4-yl-1H-pyrazolo[3,4-b]pyridin-3-amine,
N-[6-(2-Furyl)-5-pyrimidin-4-yl-1H-pyrazolo[3,4-b]pyridin-3-yl]acetamide,
5-(2-Furyl)-6-pyrimidin-4-yl-1,3-dihydro-2H-imidazo[4,5-b]pyridin-2-one
2-(2-Thienyl)-3,4′-bipyridine-5,6-diamine,
2-(2-Furyl)-3,4′-bipyridine-5,6-diamine,
6-(2-Furyl)-5-[2-(methylthio)pyrimidin-4-yl]pyridine-2,3-diamine,
6-(2-Furyl)-5-pyrimidin-4-ylpyridine-2,3-diamine,
6-Pyridin-4-yl-5-(2-thienyl)-1,3-dihydro-2H-imidazo[4,5-b]pyridin-2-one,
2-Ethoxy-5-(2-furyl)-6-pyrimidin-4-yl-3H-imidazo[4,5-b]pyridine
5-(2-Furyl)-6-pyrimidin-4-yl-3H-imidazo[4,5-b]pyridine
5-(2-Furyl)-2-methyl-6-pyrimidin-4-yl-3H-imidazo[4,5-b]pyridine,
5-(2-Furyl)-2-methyl-6-pyridin-4-yl-3H-imidazo[4,5-b]pyridine
2-Cyclopropyl-5-(2-furyl)-6-pyrimidin-4-yl-3H-imidazo[4,5-b]pyridine,
2-Cyclopropyl-5-(2-furyl)-6-pyridin-4-yl-3H-imidazo[4,5-b]pyridine,
5-(2-Furyl)-6-pyridin-4-yl-3H-imidazo[4,5-b]pyridine,
5-(2-Furyl)-6-[2-(methylthio)pyrimidin-4-yl]-3H-imidazo[4,5-b]pyridine,
5-(2-Furyl)-1-methyl-6-pyrimidin-4-yl-1,3-dihydro-2H-imidazo[4,5-b]pyridin-2-one,
6-(2-Furyl)-5-pyrimidin-4-yl-1H-pyrazolo[3,4-b]pyridine
3-Chloro-6-(2-furyl)-5-pyrimidin-4-yl-1H-pyrazolo[3,4-b]pyridine,
3-Ethoxy-6-(2-furyl)-5-pyrimidin-4-yl-1H-pyrazolo[3,4-b]pyridine,
6-(2-Furyl)-5-[2-(methylthio)pyrimidin-4-yl]-1H-pyrazolo[3,4-b]pyridin-3-amine,
6-(2-Furyl)-5-pyrimidin-4-yl-1,2-dihydro-3H-pyrazolo[3,4-b]pyridin-3-one,
6-(2-Furyl)-5-[2-(methylthio)pyrimidin-4-yl]-1H-pyrazolo[3,4-b]pyridine,
6-(2-Furyl)-5-(2-methoxypyrimidin-4-yl)-1H-pyrazolo[3,4-b]pyridine
N-Cyclopropyl-4-[6-(2-furyl)-1H-pyrazolo[3,4-b]pyridin-5-yl]pyrimidin-2-amine,
4-[6-(2-Furyl)-1H-pyrazolo[3,4-b]pyridin-5-yl]-N-isopropylpyrimidin-2-amine
5-(2-Ethoxypyrimidin-4-yl)-6-(2-furyl)-1H-pyrazolo[3,4-b]pyridine,
6-(2-Furyl)-5-(2-isopropoxypyrimidin-4-yl)-1H-pyrazolo[3,4-b]pyridine,
5-[2-(Cyclohexyloxy)pyrimidin-4-yl]-6-(2-furyl)-1H-pyrazolo[3,4-b]pyridine
6-(2-Furyl)-N-isobutyl-5-pyrimidin-4-yl-1H-pyrazolo[3,4-b]pyridin-3-amine,
N-{6-(2-Furyl)-5-[2-(methylthio)pyrimidin-4-yl]-1H-pyrazolo[3,4-b]pyridin-3-yl}acetamide,
6-(3-Fluorophenyl)-5-pyrimidin-4-yl-1H-pyrazolo[3,4-b]pyridin-3-amine
6-(3-Fluorophenyl)-5-pyrimidin-4-yl-1H-pyrazolo[3,4-b]pyridine
6-(2-Furyl)-5-pyrimidin-4-yl-1H-pyrrolo[2,3-b]pyridine
2-(3-Fluorophenyl)-6-(2-furyl)-5-pyrimidin-4-yl-1H-pyrrolo[2,3-b]pyridine
6-(2-Furyl)-2-phenyl-5-pyrimidin-4-yl-1H-pyrrolo[2,3-b]pyridine,
6-(5-Bromo-2-furyl)-3-chloro-5-pyrimidin-4-yl-1H-pyrazolo[3,4-b]pyridine,
5-(5-Bromo-2-furyl)-6-pyrimidin-4-yl-1,3-dihydro-2H-imidazo[4,5-b]pyridin-2-one,
6-(2-Furyl)-5-pyrimidin-4-yl-1H-pyrazolo[3,4-b]pyridin-3-amine, and
N-[6-(2-Furyl)-5-pyrimidin-4-yl-1H-pyrazolo[3,4-b]pyridin-3-yl]acetamide.

13. A compound of formula (I) 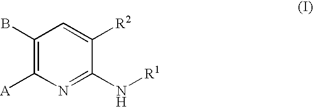
wherein:
A represents an optionally substituted monocyclic or polycyclic aryl or heteroaryl group,
B represents an optionally substituted monocyclic nitrogen-containing heterocyclic group,
and either
a) R¹ represents a hydrogen atom and R² represents a group chosen from —NH₂ and optionally substituted alkynyl groups,
or
b) R², R¹ and the —NH— group to which R¹ is attached, form a moiety chosen from the moieties of formulae (IIa), (IIb), (IIc) and (IId): 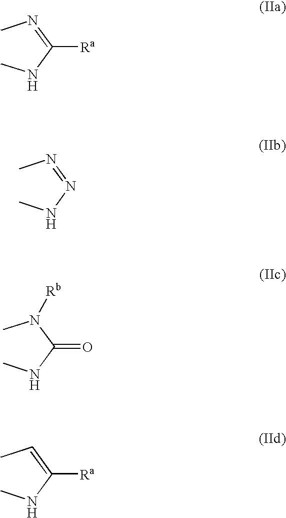
wherein:
R^{a} is a group chosen from a hydrogen atom, halogen atoms and groups chosen from optionally substituted alkyl, optionally substituted cycloalkyl, optionally substituted aryl, optionally substituted heteroaryl, —OR³, —SR³, —COOR³, —CONR³R⁴, —NR³R⁴, —NR³COR⁴ and —CN groups, wherein R³ and R⁴ are independently chosen from a hydrogen atoms and lower alkyl groups or cycloalkyl groups, and
R^{b} is a group chosen from a hydrogen atom and groups chosen from optionally substituted alkyl, optionally substituted cycloalkyl, optionally substituted aryl and optionally substituted heteroaryl groups.

14. A compound according to claim 13, wherein B represents an optionally substituted monocyclic, six-membered heterocyclic ring having one or two nitrogen atoms.

15. A compound according to claim 13, wherein B represents a group chosen from optionally substituted pyridines, optionally substituted pyrimidines, optionally substituted pyridazines and optionally substituted pyridinones.

16. A compound according to claim 13, wherein the group B is unsubstituted or substituted with one group chosen from —OR³, —SR³, —R³ and —NHR³.

17. A compound according to claim 13, wherein A represents an optionally substituted phenyl, furyl or thienyl group.

18. A compound according to claim 13, wherein the group A is unsubstituted or substituted with one group chosen from halogen atoms and lower alkyl groups.

19. A compound according to claim 13, wherein B represents a pyrimidinyl group and A represents a furyl group.

20. A compound according to claim 13, wherein either R¹ represents a hydrogen atom, or R², R¹ and the —NH— group to which R¹ is attached, form a moiety of formulae (IIc).

21. A compound according to claim 13, wherein R² represents an —NH₂ group or an optionally substituted alkynyl group.

22. A compound according to claim 13, wherein R^{a} is chosen from lower alkyl groups and cycloalkyl groups.

23. A compound according to claim 13, wherein R^{b} is chosen from lower alkyl groups and hydrogen atoms.

24. A compound according to claim 13 chosen from:
2-(3-Fluorophenyl)-3,4′-bipyridine-5,6-diamine
5-(3-Fluorophenyl)-6-pyridin-4-yl-3H-imidazo[4,5-b]pyridine,
5-(3-Fluorophenyl)-2-methyl-6-pyridin-4-yl-3H-imidazo[4,5-b]pyridine
2-Cyclopropyl-5-(3-fluorophenyl)-6-pyridin-4-yl-3H-imidazo[4,5-b]pyridine,
2-Ethyl-5-(3-fluorophenyl)-6-pyridin-4-yl-3H-imidazo[4,5-b]pyridine.
5-(3-Fluorophenyl)-6-pyridin-4-yl-3H-[1,2,3]triazolo[4,5-b]pyridine
5-(3-Fluorophenyl)-6-pyridin-4-yl-1,3-dihydro-2H-imidazo[4,5-b]pyridin-2-one,
5-Ethynyl-2-(3-fluorophenyl)-3,4′-bipyridin-6-amine,
6-(3-Fluorophenyl)-5-pyridin-4-yl-1H-pyrrolo[2,3-b]pyridine,
5-(2-Furyl)-6-pyrimidin-4-yl-1,3-dihydro-2H-imidazo[4,5-b]pyridin-2-one
2-(2-Thienyl)-3,4′-bipyridine-5,6-diamine,
2-(2-Furyl)-3,4′-bipyridine-5,6-diamine,
6-(2-Furyl)-5-[2-(methylthio)pyrimidin-4-yl]pyridine-2,3-diamine,
6-(2-Furyl)-5-pyrimidin-4-ylpyridin-2,3-diamine,
6-Pyridin-4-yl-5-(2-thienyl)-1,3-dihydro-2H-imidazo[4,5-b]pyridin-2-one,
2-Ethoxy-5-(2-furyl)-6-pyrimidin-4-yl-3H-imidazo[4,5-b]pyridine,
5-(2-Furyl)-6-pyrimidin-4-yl-3H-imidazo[4,5-b]pyridine,
5-(2-Furyl)-2-methyl-6-pyrimidin-4-yl-3H-imidazo[4,5-b]pyridine,
5-(2-Furyl)-2-methyl-6-pyridin-4-yl-3H-imidazo[4,5-b]pyridine,
2-Cyclopropyl-5-(2-furyl)-6-pyrimidin-4-yl-3H-imidazo[4,5-b]pyridine,
2-Cyclopropyl-5-(2-furyl)-6-pyridin-4-yl-3H-imidazo[4,5-b]pyridine,
5-(2-Furyl)-6-pyridin-4-yl-3H-imidazo[4,5-b]pyridine,
5-(2-Furyl)-6-[2-(methylthio)pyrimidin-4-yl]-3H-imidazo[4,5-b]pyridine,
5-(2-Furyl)-1-methyl-6-pyrimidin-4-yl-1,3-dihydro-2H-imidazo[4,5-b]pyridin-2-one,
6-(2-Furyl)-5-pyrimidin-4-yl-1H-pyrrolo[2,3-b]pyridine,
2-(3-Fluorophenyl)-6-(2-furyl)-5-pyrimidin-4-yl-1H-pyrrolo[2,3-b]pyridine,
6-(2-furyl)-2-phenyl-5-pyrimidin-4-yl-1H-pyrrolo[2,3-b]pyridine, and
5-(5-Bromo-2-furyl)-6-pyrimidin-4-yl-1,3-dihydro-2H-imidazo[4,5-b]pyridin-2-one.

25. A pharmaceutical composition comprising a compound according to claim 13 and pharmaceutically acceptable diluent or carrier.

26. A method according to claim 1, wherein the pathological condition or disease is asthma, bronchoconstriction, allergic diseases, hypertension, atherosclerosis, reperfusion injury, myocardial ischemia, retinopathy, inflammation, gastrointestinal tract disorders, cell proliferation disorders, diabetes mellitus, and/or autoimmune diseases.

27. A method for treating a subject afflicted with a pathological condition or disease susceptible to amelioration by antagonism of the A_{2B} adenosine receptor, which comprises administering to said subject an effective amount of a compound according to claim 13.

28. A method according to claim 27, wherein the pathological condition or disease is asthma, bronchoconstriction, allergic diseases, hypertension, atherosclerosis, reperfusion injury, myocardial ischemia, retinopathy, inflammation, gastrointestinal tract disorders, cell proliferation disorders, diabetes mellitus, and/or autoimmune diseases.
